(19) United States
(12) Patent Application Publication
(10) Pub. No.: US 2026/0363733 A1
(43) Pub. Date: Nov. 19, 2026

(54) UNIVERSAL STRUT ASSEMBLIES FOR EXTERNAL FIXATION SYSTEMS

(71) Applicant: ARTHREX, INC., Naples, FL (US)

(72) Inventor: Scott SMYTH, Naples, FL (US)

(21) Appl. No.: 19/676,895

(22) Filed: May 14, 2026

Related U.S. Application Data
(60) Provisional application No. 63/807,110, filed on May 16, 2025.
(60) Provisional application No. 63/901,913, filed on Oct. 20, 2025.
(60) Provisional application No. 64/048,205, filed on Apr. 24, 2026.

(51) Int. Cl. A61B 17/64; A61B 17/62; A61B 17/00

(52) U.S. Cl. CPC A61B 17/645; A61B 17/62; A61B 2017/00982

(57) ABSTRACT

External fixation systems include length-adjustable strut assemblies for stabilizing fractures and/or correcting limb deformities. An exemplary strut assembly may include a strut body and a threaded rod assembly translatably received within the strut body to selectively vary strut length. Incremental tactile feedback mechanisms may enable repeatable length adjustments, while macro quick‑adjust mechanisms may permit rapid telescopic adjustment. The threaded rod assembly may include a selectively attachable secondary rod to extend the available length range. The strut assembly may further include one or more joint assemblies configured to couple the strut assembly to fixation platforms. The joint assemblies may provide multi‑axial articulation, selective locking, adjustable dynamization, and convertible configurations supporting both dynamic hexapod and static reduction modes. Quick‑connect mounting interfaces may facilitate rapid attachment and detachment. Collectively, the strut assemblies provide modularity, precise control, and adaptable rigidity to support efficient surgical setup and optimized post‑operative bone healing.

### CROSS-REFERENCE TO RELATED APPLICATIONS

This application claims the benefit of United States Provisional Application No. 63/807,110, which was filed on May 16, 2025.

The application further claims the benefit of United States Provisional Application No. 63/901,913, which was filed on Oct. 20, 2025.

This application further claims the benefit of United States Provisional Application No. 64/048,205, which was filed on Apr. 24, 2026.

The disclosures of each of the above applications are incorporated herein by reference in their entirety.

### BACKGROUND

This disclosure relates generally to external fixation systems, and more particularly to length-adjustable universal strut assemblies for use with external fixation systems.

External fixation systems are surgical devices used to stabilize complex fractures and/or correct limb deformities. Some external fixation systems utilize rings or platforms that are connected together by rods and/or struts.

### SUMMARY

This disclosure is directed to external fixation systems for performing orthopedic surgeries for stabilizing complex fractures and/or correcting limb deformities. One or more length-adjustable universal strut assemblies may connect between a pair of platforms or rings of the external fixation system.

An exemplary strut assembly for an external fixation system may include, inter alia, a strut body extending along a strut axis, a joint assembly connected to the strut body, and a dynamization mechanism operably arranged between the strut body and the joint assembly and configured to selectively control axial compliance of the strut assembly along the strut axis. The dynamization mechanism is configurable between (i) a locked mode in which the strut assembly resists an axial length change along the strut axis under load, and (ii) an unlocked mode in which the strut assembly permits a controlled axial displacement along the strut axis under load while biasing the strut assembly toward a predefined length upon a reduction of the load.

Another exemplary strut assembly for an external fixation system may include, inter alia, a strut body extending along a strut axis, a threaded rod assembly received by the strut body and movable relative to the strut body to vary an axial length of the strut assembly, and an incremental tactile feedback mechanism operably coupled to the threaded rod assembly and configured to provide a perceptible tactile feedback during incremental adjustment of the threaded rod assembly.

Another exemplary strut assembly for an external fixation system may include, inter alia, a strut body extending along a strut axis, a threaded rod assembly received by the strut body and movable relative to the strut body to selectively vary an axial length of the strut assembly, and a macro quick adjust mechanism configured to provide telescopic macro adjustment of the threaded rod assembly relative to the strut body.

Another exemplary strut assembly for an external fixation system may include, inter alia, a strut body and a threaded rod assembly translatably received within an internal cavity of the strut body to adjust an axial length of the strut assembly. The threaded rod assembly includes an outer extension tube translatably supported relative to the strut body and constrained against rotation relative to the strut body, and an inner rod rotationally supported relative to the outer extension tube and constrained against axial movement relative to the outer extension tube.

Another exemplary strut assembly for an external fixation system may include, inter alia, a strut body, a threaded rod assembly translatably received within an internal cavity of the strut body to adjust an axial length of the strut assembly, and a joint assembly connected to the threaded rod assembly or the strut body. The joint assembly includes a ball joint configured to permit angular articulation, and a locking mechanism operably associated with the ball joint and configured to selectively restrict relative angular movement of the ball joint.

Yet another exemplary strut assembly for an external fixation system may include, inter alia, a strut body defining a strut axis, a threaded rod assembly translatably received within an internal cavity of the strut body, and a joint assembly connected to the threaded rod assembly or the strut body and operable to selectively convert the strut assembly between a first mode in which angular articulation at the joint assembly is permitted and a second mode in which the angular articulation is restricted.

An exemplary external fixation system may include, inter alia, a first support, a first strut assembly mountable to the first support, and a quick-connect mount for detachably connecting the first strut assembly relative to the first support. The quick-connect mount includes a mount body defining a slot configured to receive the first support, a lock nut rotationally supported by the mount body, and a lock screw operably coupled to the mount body and engageable with the lock nut. Relative movement between the lock screw and the lock nut captures the first support within the slot to secure the first strut assembly to the first support.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 is a perspective view of an exemplary external fixation system including strut assemblies between supports according to an implementation.

FIG. 2 illustrates the external fixation system of FIG. 1 arranged relative to a bone.

FIG. 3 illustrates an exemplary strut assembly including a dynamization mechanism.

FIG. 4 illustrates the dynamization mechanism of FIG. 3 in a locked position.

FIG. 5 is a cross-sectional view of the dynamization mechanism taken along line 5-5 of FIG. 4.

FIG. 6A is a perspective view of the dynamization mechanism of FIG. 3 in the locked position.

FIG. 6B is another perspective view of the dynamization mechanism of FIG. 3 in an unlocked position.

FIG. 7 illustrates the dynamization mechanism of FIG. 3 in the unlocked position and the strut assembly in a collapsed state.

FIGS. 8, 9, and 10 illustrate an exemplary incremental tactile feedback mechanism of a strut assembly.

FIGS. 11, 12, and 13 illustrate another exemplary incremental tactile feedback mechanism.

FIGS. 14, 15, 16, and 17 illustrate an exemplary macro quick adjustment of a strut assembly.

FIGS. 18 and 19 illustrate a lock knob of the macro quick adjust mechanism of FIGS. 14-17.

FIGS. 20, 21, 22, and 23 illustrate an exemplary threaded rod assembly of a strut assembly.

FIGS. 24 and 25 illustrate an outer extension tube of the threaded rod assembly of FIGS. 20-23.

FIGS. 26, 27, 28, and 29 illustrate a secondary rod of the threaded rod assembly of FIGS. 20-23.

FIGS. 30, 31, 32, and 33 illustrate an exemplary method for connecting a secondary rod to a threaded rod assembly of a strut assembly.

FIGS. 34, 35, 36, and 37 illustrate another exemplary threaded rod assembly for a strut assembly.

FIGS. 38, 39, 40, 41, and 42 illustrate an exemplary joint assembly of a strut assembly.

FIG. 43 illustrates another exemplary joint assembly of a strut assembly.

FIG. 44 illustrates another exemplary joint assembly of a strut assembly.

FIG. 45 illustrates another exemplary joint assembly of a strut assembly.

FIGS. 46, 47, 48, 49, 50, 51, 52, and 53 illustrate yet another exemplary joint assembly of a strut assembly.

FIGS. 54, 55, 56, 57, and 58 illustrate an exemplary quick connect mount for mounting a strut assembly to a platform of an external fixation system.

### DETAILED DESCRIPTION

This disclosure is directed to external fixation systems for performing orthopedic surgeries for stabilizing complex fractures and/or correcting limb deformities. The external fixation systems may include one or more universal strut assemblies that connect between a pair of platforms of the external fixation system.

The external fixation systems described herein may be used to stabilize complex fractures and/or correct limb deformities. The external fixation systems could be configured as a hexapod-type dynamic external fixation system, an Ilizarov-type static external fixation system, or any other type of external fixation system and can be arranged about and secured to one or more bones of a human musculoskeletal system. The bone(s) may be part of any limb or joint of the human musculoskeletal system, including but not limited to that of the leg, hand, wrist, forearm, foot, ankle, etc.

An exemplary external fixation system may include a plurality (e.g., two or more) of platforms. The platforms may alternatively be referred to as plates or rings. Each platform of the system may be either a full ring or a partial ring and can be arranged to circumferentially extend at least partially around the bone(s).

Adjacent pairs of platforms of the external fixation system can be connected together by one or more length-adjustable strut assemblies. Each platform may include openings for receiving portions of the strut assemblies. The strut assemblies may connect between fixation points of the external fixation system to allow for precise adjustment of the system relative to the bone(s).

An exemplary strut assembly for the external fixation system may include a strut body, a threaded rod assembly translatably received within an internal cavity of the strut body, a first joint assembly connected to an end of the strut body and configured for securing the strut assembly to a first platform of the external fixation system, and a second joint assembly connected to an end of the threaded rod assembly and configured for securing the strut assembly to a second platform of the external fixation system.

The strut assembly may include a dynamization mechanism for achieving target dynamization. The dynamization mechanism may include a dynamization adjustment knob that is located outside of the strut body, a cross pin, and a spring. The dynamization adjustment knob may be turned to either lock or unlock the dynamization.

The strut assembly may include an incremental tactile feedback mechanism for incrementally adjusting a length of the strut assembly (e.g., by translating the threaded rod assembly relative to the strut body). The incremental tactile feedback mechanism may include a quick adjust knob that is rotatable relative to a spring collar for performing precise incremental (e.g., ¼ mm increments, etc.) adjustments to the length of the strut assembly. The quick adjust knob and the spring collar may engage/disengage one another at multiple ramped tab/slot interfaces.

The incremental tactile feedback mechanism may include a cam follower assembly established by a quick adjust housing (e.g., the cam) and a cam adjustment collar (e.g., the follower).

The strut assembly may include a macro quick adjust mechanism that enables rapid, tool-free telescopic macro adjustments to the length of the strut assembly. The macro quick adjust mechanism may include a first quick adjust button, a second quick adjust button, a spring and pin assembly arranged between the first and second quick adjust buttons, and a set screw removably arranged between the first and second quick adjust buttons. The macro quick adjust mechanism may be unlocked by removing, unthreading, or otherwise disengaging the set screw. The first and second quick adjust buttons may then be pressed toward one another to disengage threads of each button from corresponding threads/engagement features of an outer extension tube of the threaded rod assembly, thereby permitting the threaded rod assembly to translate/telescope relative to the strut body. A subsequent release of the first and second quick adjust buttons causes the threads to reengage those of the outer extension tube to maintain the current length of the strut assembly.

The macro quick adjust mechanism may include a lockout mechanism that is arranged to lock out the macro quick adjust mechanism so that macro adjustments can be made without the need to press the first and second quick adjust buttons.

The threaded rod assembly of the strut assembly may include an outer extension tube, an inner threaded rod received through a cannulation of the outer extension tube, and a secondary rod that is connectable to the outer extension tube for increasing the length of the threaded rod assembly, such as for accommodating larger anatomies, for example. The secondary rod and the outer extension tube may each include tangs/orientation features for facilitating connection of the secondary rod to the outer extension tube. A threaded end portion of the inner threaded rod may screw into the secondary rod upon a clockwise rotation of a hex nut of the second joint assembly.

The joint assemblies of the strut assembly may each include a cannulated strut-to-platform mount mechanism that enables adjustable post-operative dynamization, locking functionality, and maximum range of motion. The cannulated strut-to-platform mount mechanism may include a cannulated ring mount base, a swivel lock cap that receives a ball arm that is connected to either the strut body or the threaded rod assembly, a threaded socket, a detent, a lock screw, and a spring arranged between the detent and the lock screw.

A taper lock may lock rotation of the swivel lock cap relative to the ring mount base. The ball arm-to-swivel lock cap interface can allow for 360 degrees of rotation and 180 degrees of swivel when the respective joint assembly is joined to the platform.

The detent can be biased toward the ball member of the ball arm and may be accommodated within a recessed feature of the ball member to allow the strut axis to be held orthogonal to the plane of the platform. Turning the lock screw in a first direction can lock movement of the ball member relative to the swivel lock cap, and turning the lock screw in a second direction can allow movement of the ball member relative to the swivel lock cap.

The cannulated strut-to-platform mount mechanism may include a cannulated ring mount base that includes internal threads for receiving a cannulated screw. A finger screw may thread into the cannulated screw for moving a detent toward or away from a ball member that is received by a swivel lock cap that connects to the ring mount base.

The cannulated strut-to-platform mount mechanism may include a base post received within the cannulated ring mount base. A thumb screw can be turned to move the base post toward or away from the ball joint interface for locking/unlocking movement.

The cannulated strut-to-platform mount mechanism may include a swivel body, a lock body, a threaded ball interface, and a screw. The lock body may be turned to lock or unlock a ball member relative to the threaded ball interface. Dynamization can be achieved by turning the lock body counterclockwise such that the threaded ball interface moves towards a bottom of the screw.

The first and second joint assemblies of the strut assembly may each include a convertible strut-to-platform mount mechanism that includes a swivel housing that is connected to either the strut body or the threaded rod assembly, a ball arm that interfaces with the swivel housing, a strut base, an internal spring, and a cross pin that secures the strut base to the ball arm. The strut base may be toggled to convert the strut assembly from a first mode (e.g., dynamic mode) to a second mode (e.g., static reduction mode).

Any of the joint assembly designs discussed above can be incorporated into a quick connect ring mount assembly that includes a quick connect mechanism for connecting/disconnecting the strut assembly to one of the platforms of the external fixation system. These and other features of this disclosure are further detailed below.

An exemplary strut assembly for an external fixation system may include, inter alia, a strut body extending along a strut axis, a joint assembly connected to the strut body, and a dynamization mechanism operably arranged between the strut body and the joint assembly and configured to selectively control axial compliance of the strut assembly along the strut axis. The dynamization mechanism is configurable between (i) a locked mode in which the strut assembly resists an axial length change along the strut axis under load, and (ii) an unlocked mode in which the strut assembly permits a controlled axial displacement along the strut axis under load while biasing the strut assembly toward a predefined length upon a reduction of the load.

In any implementations, the dynamization mechanism includes a dynamization shaft and a dynamization adjustment control that is operably connected to the dynamization shaft.

In any implementations, a first end of the dynamization shaft is secured to the joint assembly, and a second end of the dynamization shaft is secured to a coupler that is secured to the strut body.

In any implementations, the first end of the dynamization shaft is secured to a swivel housing of the joint assembly.

In any implementations, the dynamization adjustment control includes a user-actuatable adjustment knob that is rotatable to transition the dynamization mechanism between the locked mode and the unlocked mode.

In any implementations, the user-actuatable adjustment knob threadably engages the dynamization shaft.

In any implementations, rotation of the user-actuatable adjustment knob about the strut axis is configured to vary the controlled axial displacement in the unlocked mode.

In any implementations, the dynamization mechanism includes a biasing member arranged to urge the strut assembly toward the predefined length upon the reduction of the load when the dynamization mechanism is in the unlocked mode.

In any implementations, the biasing member is arranged between a dynamization adjustment control and a coupler of the dynamization mechanism.

In any implementations, the dynamization mechanism includes a retention pin movable within a retention slot extending parallel to the strut axis.

In any implementations, the retention pin secures a coupler of the dynamization mechanism to the strut body, and the retention slot is formed in the strut body.

In any implementations, the retention slot is sized to delimit a maximum permitted axial displacement of the strut assembly in the unlocked mode.

In any implementations, the retention pin is configured to engage an end of the retention slot to limit axial displacement when the maximum permitted axial displacement is reached.

In any implementations, the controlled axial displacement is enabled in the unlocked mode without removal of the strut assembly from the external fixation system.

In any implementations, the locked mode is configured for use during an initial post‑operative fixation phase and the unlocked mode is configured for use during a subsequent bone‑healing phase.

Another exemplary strut assembly for an external fixation system may include, inter alia, a strut body extending along a strut axis, a threaded rod assembly received by the strut body and movable relative to the strut body to vary an axial length of the strut assembly, and an incremental tactile feedback mechanism operably coupled to the threaded rod assembly and configured to provide a perceptible tactile feedback during incremental adjustment of the threaded rod assembly.

In any implementations, the perceptible tactile feedback occurs at each of a plurality of discrete adjustment increments during relative movement between the threaded rod assembly and the strut body.

In any implementations, each perceptible tactile feedback event corresponds to an incremental change in the axial length of the strut assembly.

In any implementations, the incremental tactile feedback mechanism includes a plurality of ramped engagement features circumferentially spaced to define the plurality of discrete adjustment increments.

In any implementations, the plurality of ramped engagement features include ramp tabs configured to engage corresponding ramp slots.

In any implementations, the incremental tactile feedback mechanism includes a quick‑adjust knob operably coupled to the threaded rod assembly and a spring collar movably mounted to the strut body.

In any implementations, each incremental adjustment corresponds to approximately one-quarter turn of the quick-adjust knob relative to the strut body.

In any implementations, the spring collar is biased toward the quick‑adjust knob by at least one biasing member.

In any implementations, the incremental tactile feedback mechanism comprises a cam and a follower configured to generate the perceptible tactile feedback during the incremental adjustment.

In any implementations, the incremental tactile feedback mechanism comprises a quick-adjust housing that functions as the cam and a cam adjustment collar that functions as the follower.

In any implementations, the cam adjustment collar is biased toward the quick-adjust housing by a biasing member.

In any implementations, the quick-adjust housing and the cam adjustment collar provide a plurality of ramped engagement features circumferentially spaced to define discrete adjustment increments.

In any implementations, the cam adjustment collar includes a guide feature configured to prevent relative rotation with respect to the strut body while permitting axial translation along the strut body.

In any implementations, the guide feature includes a projection formed on the cam adjustment collar and received within a corresponding slot formed in the strut body.

In any implementations, the perceptible tactile feedback includes an audible indication of the incremental adjustment.

Another exemplary strut assembly for an external fixation system may include, inter alia, a strut body extending along a strut axis, a threaded rod assembly received by the strut body and movable relative to the strut body to selectively vary an axial length of the strut assembly, and a macro quick adjust mechanism configured to provide telescopic macro adjustment of the threaded rod assembly relative to the strut body.

In any implementations, the macro quick adjust mechanism enables rapid axial repositioning of the threaded rod assembly relative to the strut body without requiring incremental rotation of the threaded rod assembly.

In any implementations, the macro quick adjust mechanism includes a first quick adjust button, a second quick adjust button, and a spring.

In any implementations, the spring resiliently biases the first quick adjust button and the second quick adjust button toward an engaged state with the threaded rod assembly.

In any implementations, the macro quick adjust mechanism further includes a set screw configured to selectively restrict movement between the first quick adjust button and the second quick adjust button.

In any implementations, the first quick adjust button and the second quick adjust button are selectively movable toward one another to release the threaded rod assembly and thereby permit the telescopic macro adjustment of the threaded rod assembly relative to the strut body.

In any implementations, release of the first quick adjust button and the second quick adjust button results in automatic re‑engagement of the buttons with the threaded rod assembly to retain the strut assembly at a selected axial length.

In any implementations, the first quick adjust button and the second quick adjust button are operably coupled by at least one lock pin.

In any implementations, the spring is received over a pin that is mounted to the first quick adjust button.

In any implementations, the macro quick adjust mechanism includes a lock knob movable between a locked state that inhibits the telescopic macro adjustment and an unlocked state that permits the telescopic macro adjustment.

Another exemplary strut assembly for an external fixation system may include, inter alia, a strut body and a threaded rod assembly translatably received within an internal cavity of the strut body to adjust an axial length of the strut assembly. The threaded rod assembly includes an outer extension tube translatably supported relative to the strut body and constrained against rotation relative to the strut body, and an inner rod rotationally supported relative to the outer extension tube and constrained against axial movement relative to the outer extension tube.

In any implementations, the threaded rod assembly includes an anti-rotation pin that engages a flat section of the outer extension tube to inhibit the rotation of the outer extension tube relative to the strut body.

In any implementations, the threaded rod assembly includes a coupling member that rotationally supports the inner rod relative to the outer extension tube.

In any implementations, the coupling member includes a pin.

In any implementations, the inner rod includes a threaded end portion that projects outwardly away from an end of the outer extension tube.

In any implementations, a coupling fastener is mounted to the inner rod at an opposite end from the threaded end portion and is configured to impart rotational motion to the inner rod relative to the outer extension tube.

In any implementations, the inner rod includes a shaft portion and a ball portion.

In any implementations, the threaded rod assembly further includes a secondary rod selectively attachable to the inner rod to increase an available axial adjustment range of the strut assembly.

In any implementations, the secondary rod is attachable to a threaded end portion of the inner rod.

In any implementations, the secondary rod and the outer extension tube include cooperating mating features configured to align the secondary rod to the threaded rod assembly during attachment.

In any implementations, the cooperating mating features include tangs on one of the secondary rod or the outer extension tube and corresponding slots on the other of the secondary rod or the outer extension tube.

In any implementations, a modular indicator member is selectively attachable to the inner rod or the secondary rod to indicate a strut length configuration.

In any implementations, the modular indicator member is selectively attachable to the inner rod to indicate a first strut length configuration and is selectively repositionable for attachment to the secondary rod to indicate a second strut length configuration.

In any implementations, the modular indicator member includes an indicator pointer that aligns with a first set of length designations formed on the strut body to indicate the first strut length configuration when the modular indicator member is attached to the inner rod and that further aligns with a second set of length designations formed on the strut body to indicate the second strut length configuration when the modular indicator member is attached to the secondary rod.

In any implementations, a fastener secures the modular indicator member to the secondary rod.

Another exemplary strut assembly for an external fixation system may include, inter alia, a strut body, a threaded rod assembly translatably received within an internal cavity of the strut body to adjust an axial length of the strut assembly, and a joint assembly connected to the threaded rod assembly or the strut body. The joint assembly includes a ball joint configured to permit angular articulation, and a locking mechanism operably associated with the ball joint and configured to selectively restrict relative angular movement of the ball joint.

In any implementations, the locking mechanism is adjustable between an unlocked state permitting angular articulation of the ball joint and a locked state restricting angular articulation of the ball joint.

In any implementations, the ball joint includes a swivel lock cap and a ball portion movably received by the swivel lock cap.

In any implementations, the joint assembly further includes a cannulated mount base rotatably coupled to the swivel lock cap.

In any implementations, the locking mechanism includes a threaded socket including a curved seating surface that supports the ball portion, a detent, and a lock screw rotatable to advance or retract the detent toward or away from the ball portion.

In any implementations, the locking mechanism includes a detent assembly and a finger screw rotatable to advance or retract the detent assembly toward or away from the ball portion.

In any implementations, the locking mechanism includes a lock nut, a base post operably connected to the lock nut, and a thumb screw rotatable to advance or retract the base post toward or away from the ball portion.

In any implementations, the locking mechanism includes a threaded ball interface operably connected to the swivel lock cap and a lock body rotatable to advance or retract the threaded ball interface toward or away from the ball portion.

In any implementations, the locking mechanism is configured to permit partial locking of the ball joint to allow limited angular movement under load.

In any implementations, the limited angular movement provides controlled dynamization of the strut assembly.

Yet another exemplary strut assembly for an external fixation system may include, inter alia, a strut body defining a strut axis, a threaded rod assembly translatably received within an internal cavity of the strut body, and a joint assembly connected to the threaded rod assembly or the strut body and operable to selectively convert the strut assembly between a first mode in which angular articulation at the joint assembly is permitted and a second mode in which the angular articulation is restricted.

In any implementations, the first mode corresponds to a hexapod configuration and the second mode corresponds to a static reduction configuration.

In any implementations, the joint assembly includes a swivel housing, a mounting rod including a ball portion that interfaces with the swivel housing, and a strut base that is operably coupled to both the swivel housing and the mounting rod.

In any implementations, the strut base is rotatable about the strut axis to convert the joint assembly between the first mode and the second mode.

In any implementations, the joint assembly further includes a cross pin that couples the strut base to a shaft portion of the mounting rod.

In any implementations, the strut base includes a first relief region associated with the first mode and a second relief region associated with the second mode.

In any implementations, the second relief region is deeper than the first relief region to promote engagement that restricts the angular articulation in the second mode.

In any implementations, the cross pin is positioned in the first relief region in the first mode and is positioned in the second relief region in the second mode.

In any implementations, the joint assembly further includes a biasing member arranged to urge the strut base toward the swivel housing to maintain the joint assembly in a selected one of the first mode or the second mode.

In any implementations, the biasing member is a spring that is arranged about a shaft portion of the mounting rod.

An exemplary external fixation system may include, inter alia, a first support, a first strut assembly mountable to the first support, and a quick-connect mount for detachably connecting the first strut assembly relative to the first support. The quick-connect mount includes a mount body defining a slot configured to receive the first support, a lock nut rotationally supported by the mount body, and a lock screw operably coupled to the mount body and engageable with the lock nut. Relative movement between the lock screw and the lock nut captures the first support within the slot to secure the first strut assembly to the first support.

In any implementations, disengagement of the lock screw from the lock nut permits removal of the first support from the slot.

In any implementations, the quick-connect mount further includes a biasing member adapted to urge the lock screw toward the lock nut.

In any implementations, the quick-connect mount further includes a cross pin that is secured to the lock screw.

In any implementations, the mount body includes a groove and an exit slot.

In any implementations, the cross pin is positionable in the groove to permit lateral insertion of the first support into the slot.

In any implementations, the cross pin is movable from the groove to the exit slot to enable axial movement of the lock screw toward the lock nut.

In any implementations, the quick-connect mount further includes a mount base configured to attach to a joint assembly of the first strut assembly.

In any implementations, the joint assembly includes a ball joint.

In any implementations, the lock nut is operably coupled to an upper platform of the mount body, and the lock screw is operably coupled to a lower platform of the mount body.

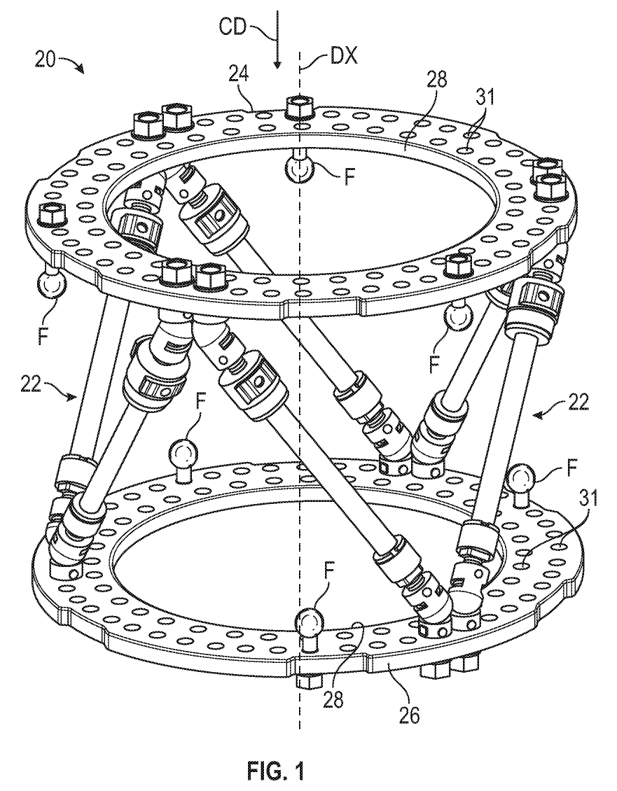

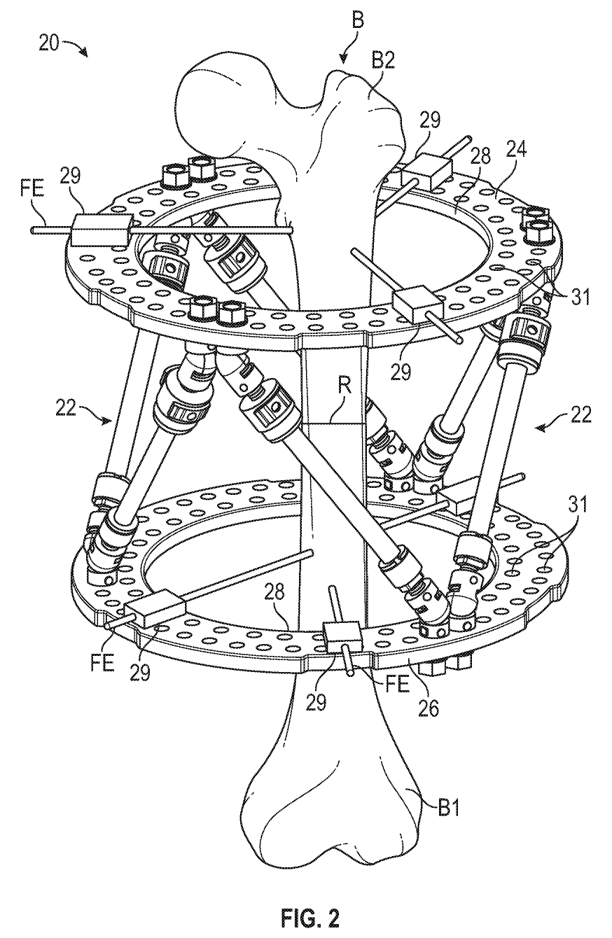

FIGS. 1–2 illustrate an exemplary external fixation system 20. The external fixation system 20 may be utilized in a variety of orthopaedic procedures, including, but not limited to, fracture stabilization procedures and bone deformity correction procedures. The external fixation system 20 may include one or more strut assemblies 22 configured to interconnect a first support 24 and a second support 26. The supports 24, 26 may also be referred to as platforms, plates, or rings. The strut assemblies 22 may be circumferentially distributed about a device axis DX (e.g., a longitudinal axis of the external fixation system 20). The supports 24, 26 may be spaced from one another and arranged along the device axis DX.

The supports 24, 26 may embody various geometries. In the implementation of FIGS. 1-2, the supports 24, 26 include a ring-shaped geometry. However, other geometries, including but not limited to partial ring geometries, may be utilized within the scope of this disclosure.

Each of the supports 24, 26 may include one or more openings 28. The openings 28 may be dimensioned to at least partially receive one or more bones B of a patient, as illustrated in FIG. 2. The bones B may include long bones, such as, for example, the femur, tibia, ulna, and/or radius. The supports 24, 26 may be fixedly attached or otherwise secured to the bone B. In some implementations, the supports 24, 26 may include one or more guide structures 29 configured to facilitate placement of respective fixation elements FE into the bone B. The fixation elements FE may include, without limitation, Kirschner wires, pins, screws, or combinations thereof. In use, a surgeon may cross‑pin or otherwise secure the external fixation system 20 to the bone B to limit relative movement therebetween.

The external fixation system 20 may further include one or more fiducials F (e.g., markers), as illustrated in FIG. 1. The fiducials F may be fixedly attached or otherwise secured to the supports 24, 26 and may have a known or identifiable geometry. The fiducials F may be configured to facilitate determination of a two‑dimensional and/or three‑dimensional orientation of the supports 24, 26 relative to the patient anatomy using one or more imaging modalities, such as fluoroscopic imaging, for example. In some implementations, the fiducials F may include a portion having a generally spherical geometry. By way of example, three fiducials F may be secured to each support 24, 26. Images acquired from two or more different viewing angles may be utilized to determine the position and orientation of the supports 24, 26 relative to the patient anatomy. The positions of the strut assemblies 22 may likewise be determined or indexed based on their respective attachment locations along the supports 24, 26.

The strut assemblies 22 may be pivotably connected to the supports 24, 26. Each support 24, 26 may include a plurality of additional openings 31, separate from openings 28, configured to receive attachment components for securing the strut assemblies 22 to the respective support 24, 26. Each strut assembly 22 may be adjustable to selectively vary a length of the strut assembly 22 and thereby adjust a distance between the supports 24, 26. In certain applications, one or more of the strut assemblies 22 may be shortened or lengthened to apply tensile or compressive forces across a fracture, osteotomy, or resection site of the bone B.

In the exemplary embodiment illustrated in FIGS. 1–2, the external fixation system 20 includes a hexapod configuration having six strut assemblies 22. In such a configuration, the strut assemblies 22 may be independently adjustable to correct multi‑planar bone deformities with six degrees of freedom. However, it should be understood that the external fixation system 20 may include more or fewer than six strut assemblies 22 without departing from the scope of this disclosure. For example, certain external fixation system implementations may include two, three, four, or five strut assemblies 22 depending on the desired application and fixation requirements.

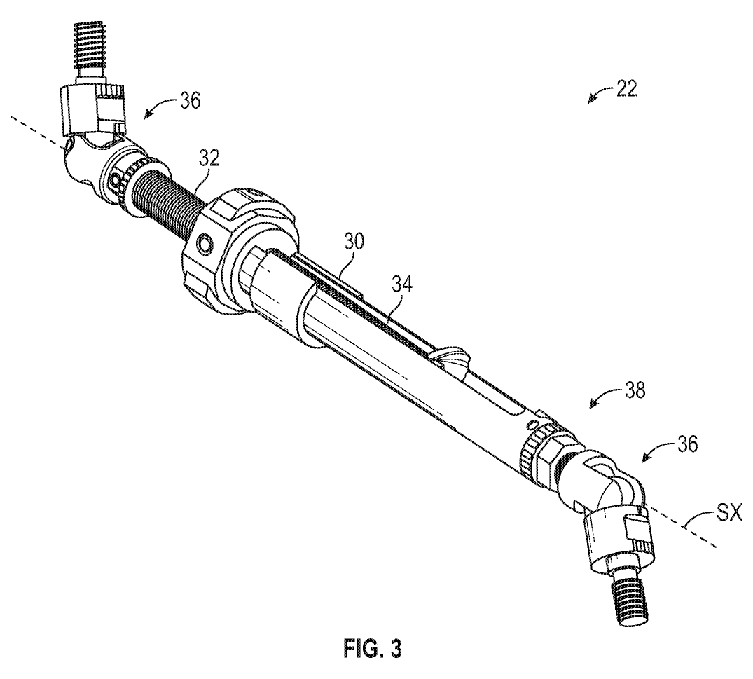

Referring now to FIG. 3, and with continuing reference to FIGS. 1–2, each strut assembly 22 may include a strut body 30 and a threaded rod assembly 32. The strut body 30 and the threaded rod assembly 32 may extend generally along a strut axis SX, which may define a longitudinal axis of the strut assembly 22. The strut axis SX may extend between opposing ends of the strut assembly 22 that are configured for connecting the strut assembly 22 to the respective supports 24, 26.

The strut assembly 22 may be length‑adjustable along the strut axis SX. In particular, the threaded rod assembly 32 may be translatably received within an internal cavity 34 of the strut body 30 such that relative axial movement between the threaded rod assembly 32 and the strut body 30 varies an overall length of the strut assembly 22. In some implementations, the threaded rod assembly 32 may include external threading configured to mate with corresponding internal threading of the strut body 30 to selectively set and maintain the strut length. As illustrated in FIG. 2, the bone B may include adjacent bone segments B1, B2 that meet at an osteotomy or resection site R, with the supports 24, 26 respectively fixed to the bone segments B1, B2. Adjustment of the length of one or more strut assemblies 22, such as by reducing the strut length, may apply a compressive force across the resection site R between the bone segments B1, B2 when the supports 24, 26 are secured to the respective bone segments.

The strut assembly 22 shown in FIG. 3 may further include one or more joint assemblies 36 positioned at opposite ends of the strut assembly 22. Each joint assembly 36 may be configured to pivotably couple the strut assembly 22 to a corresponding support 24, 26. In an exemplary implementation, each joint assembly 36 includes a ball joint configuration that permits multi‑axial articulation between the strut assembly 22 and the associated support 24, 26. The joint assemblies 36 may allow relative angular motion during installation and adjustment of the external fixation system 20. Each joint assembly 36 may further be selectively lockable to restrict or prevent relative rotation between the strut assembly 22 and the respective support 24, 26 once a desired alignment or fixation state has been achieved.

The strut assembly 22 may additionally be adjustable to establish a desired direction of collapse CD (schematically shown in FIG. 1) of the external fixation system 20. The direction of collapse CD may be selected to control mechanical dynamization of the fixation construct during postoperative bone healing or consolidation. In some implementations, the direction of collapse CD may be oriented substantially parallel to the device axis DX, thereby promoting controlled axial loading of the bone B while limiting undesirable shear forces across a fracture site, osteotomy, or resection.

The external fixation system 20 may require a relatively high degree of rigidity during an initial postoperative period to maintain accurate alignment of the adjacent bone segments B1, B2 and to prevent relative motion that could adversely influence healing. As bone consolidation progresses, however, continued rigid fixation may inhibit optimal bone remodeling and maturation. Conventional strut fixation constructs typically lack the ability to selectively transition from an initially rigid condition to a state permitting limited, controlled axial compliance without exchanging hardware or substantially reconfiguring the fixation construct. As further described below, the strut assembly 22 may therefore incorporate a dynamization mechanism 38 configured to enable the strut assembly 22 to provide initial rigid fixation and thereafter enable a controlled, adjustable degree of axial compliance, or dynamization, along the device axis DX while maintaining overall alignment and stability of the bone B.

The dynamization mechanism 38 may be configured to achieve a target or prescribed amount of dynamization during bone healing by selectively varying the axial rigidity of the strut assembly 22. In some implementations, the dynamization mechanism 38 may be adjustable to vary an amount of permitted dynamization travel, such as a controlled axial collapse and subsequent return of the strut assembly 22. The permitted dynamization travel may occur substantially along the strut axis SX and may substantially exclude radial or off‑axis movement relative to the strut axis SX. When the bone B is subjected to an external load, such as patient body weight, the dynamization mechanism 38 may allow a dimension of the strut assembly 22, such as its effective length, to temporarily decrease by the permitted amount. The permitted dynamization travel may be distinct from length adjustments of the strut assembly 22 used to correct a bone deformity and may be enabled only after completion of the deformity correction or prescription period. In some implementations, the dynamization mechanism 38 may be configured such that the permitted dynamization travel does not increase a maximum length of the strut assembly 22 previously set during adjustment.

The dynamization mechanism 38 may be adjustable using manual and/or automated (e.g., robotic) techniques. In some implementations, the dynamization mechanism 38 may incorporate a servo to drive rotation and/or axial displacement of the strut assembly 22.

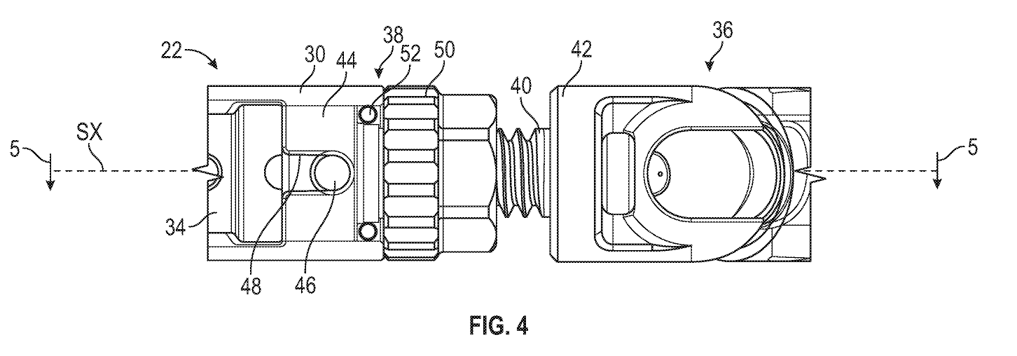

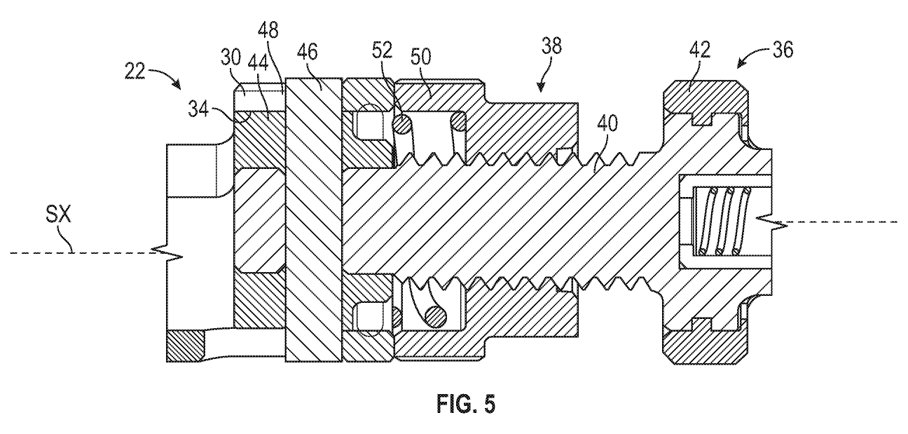

The dynamization mechanism 38 of the strut assembly 22 is further illustrated in FIGS. 4–5. The dynamization mechanism 38 may include a dynamization shaft 40 extending generally along the strut axis SX. A first end of the dynamization shaft 40 may be fixedly attached, integrally formed with, or otherwise secured to a joint body 42 (e.g., a swivel housing) of the joint assembly 36 that is coupled to the strut body 30. A second, opposite end of the dynamization shaft 40 may be operatively coupled to a coupler 44, which may be configured for securement to the strut body 30.

The coupler 44 may be at least partially received within the internal cavity 34 of the strut body 30. The coupler 44 may define an opening or other feature configured to receive a retention pin 46. The retention pin 46 may be retained within a corresponding retention slot 48 formed in the strut body 30. The retention slot 48 may extend in a direction substantially parallel to the strut axis SX and may be dimensioned to limit axial travel of the retention pin 46 along the length of the retention slot 48. In this manner, the permitted axial movement of the retention pin 46 may define and correspondingly limit relative axial movement between the strut body 30 and the joint body 42 of the joint assembly 36, thereby constraining the range of dynamization provided by the strut assembly 22.

The dynamization mechanism 38 may further include a dynamization adjustment control 50. In some implementations, the dynamization adjustment control 50 may be configured as a rotatable adjustment knob operable to selectively lock or unlock dynamization of the strut assembly 22. The dynamization adjustment control 50 may include internal or external threading adapted to engage corresponding threading of the dynamization shaft 40, thereby securing the dynamization adjustment control 50 to the strut assembly 22 and enabling controlled axial positioning of the dynamization adjustment control 50 relative to the strut body 30.

The dynamization mechanism 38 may further include a biasing member 52 positioned between the coupler 44 and the dynamization adjustment control 50. The biasing member 52 may be captured in compression and may include any suitable resilient element, including, for example, a coil spring, wave spring, or similar biasing member. The biasing member 52 may be configured to bias the strut body 30 away from the dynamization adjustment control 50 along the strut axis SX, thereby urging the strut assembly 22 substantially toward a preset or original length, such as a length established at the end of a deformity‑correction or prescription phase, when the strut assembly 22 is unloaded. For purposes of this disclosure, the term “substantially” means ±2 percent of the stated value or relationship unless otherwise indicated. Adjustment of the dynamization adjustment control 50 may vary the compression of the biasing member 52 and thereby adjust the axial compliance of the strut assembly 22.

The dynamization adjustment control 50 may be selectively movable, such as by rotation about the strut axis SX, to vary or set an amount of permitted dynamization travel of the strut assembly 22. The permitted dynamization travel may be continuously variable or may correspond to one or more discrete settings. In some implementations, the dynamization adjustment control 50 and/or the strut body 30 may include indicia, such as laser‑etched marks, corresponding to respective dynamization settings to enable a surgeon or other clinical user to visually confirm implementation of a desired or prescribed dynamization protocol.

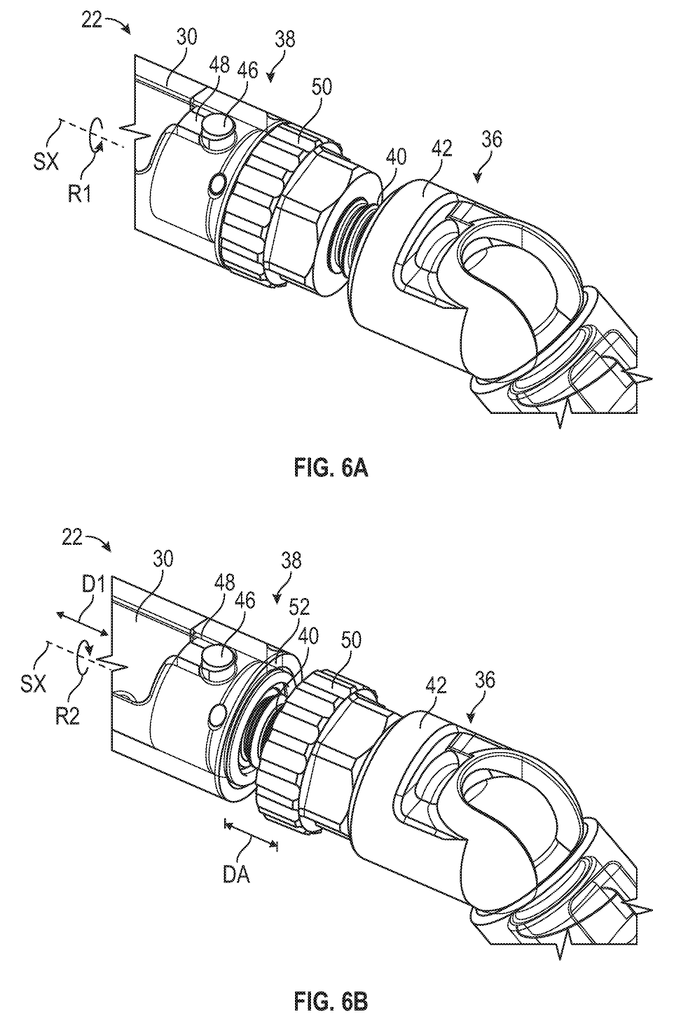

Referring now to FIGS. 6A–6B, with continuing reference to FIGS. 3–5, the dynamization mechanism 38 may be selectively operable between a locked mode and an unlocked (dynamization) mode. In the locked mode, schematically illustrated in FIG. 6A, the dynamization mechanism 38 may constrain relative axial movement such that the strut body 30 is substantially immovable relative to the dynamization shaft 40, thereby maintaining a fixed length of the strut assembly 22. In the unlocked mode, schematically illustrated in FIG. 6B, the strut body 30 may be permitted to move axially relative to the dynamization shaft 40 to allow controlled dynamization of the strut assembly 22. In the unlocked mode, the dynamization adjustment control 50 may be selectively repositioned to vary an amount of permitted dynamization travel, such as controlled axial collapse and return, of the strut assembly 22.

When a patient applies a load to the surgically treated limb, such as during weight‑bearing activities, the strut assembly 22 may be permitted to collapse when the dynamization mechanism 38 is in the unlocked mode. The extent of such collapse may be limited to a predefined amount of permitted dynamization travel set by the dynamization adjustment control 50. Adjustment of the dynamization adjustment control 50 may therefore be used to selectively vary, such as by increasing or decreasing, the amount of permitted axial collapse and subsequent return of the strut assembly 22. In some implementations, the dynamization mechanism 38 may be adjusted progressively over a prescribed period of time to gradually transfer a portion of the load from the external fixation system 20 to the corrected bone B as healing progresses.

The dynamization adjustment control 50 may be configured to move relative to the dynamization shaft 40 to selectively lock or unlock dynamization of the strut assembly 22 when transitioning between the locked mode and the unlocked mode. In the unlocked mode, the position of the dynamization adjustment control 50 may be adjustable to set an axial rigidity of the strut assembly 22 and thereby define the permitted amount of dynamization travel. The dynamization adjustment control 50 may be rotatable about, and/or axially translatable along, the strut axis SX to vary its position. In some implementations, rotation of the dynamization adjustment control 50 in a first direction R1 (e.g., counterclockwise) about the strut axis SX may transition the dynamization mechanism 38 from the locked mode to the unlocked mode, while rotation in a second, opposite direction R2 (e.g., clockwise) may transition the dynamization mechanism 38 from the unlocked mode to the locked mode.

When the dynamization mechanism 38 is set in the unlocked mode, the dynamization adjustment control 50 may occupy any of a range of positions relative to the dynamization shaft 40. In a locked position associated with the locked mode, a body of the dynamization adjustment control 50 may abut an end of the strut body 30, thereby preventing relative axial movement between the strut body 30 and the dynamization shaft 40. In the unlocked mode, the strut body 30 may be movable relative to the dynamization shaft 40 along the strut axis SX. In particular, the strut body 30 may translate in an axial direction D1 to vary its position along the strut axis SX. In a fully unlocked position associated with a maximum permitted dynamization travel, the body of the dynamization adjustment control 50 may be positioned to abut the joint body 42 of the joint assembly 36. In the unlocked mode, the strut body 30 may be configured to freely translate along the dynamization shaft 40 by a limited amount, thereby permitting controlled collapse and subsequent expansion of the strut assembly 22.

A maximum permitted amount of dynamization travel of the strut assembly 22 may be defined by a distance DA along the strut axis SX. The distance DA may be set in response to adjustment of the dynamization adjustment control 50 and may extend between the strut body 30 and the joint body 42 when the retention pin 46 abuts an end of the retention slot 48, as illustrated in FIG. 6B. In some implementations, the strut body 30 may be configured to translate freely along the strut axis SX through a maximum distance substantially equal to the distance DA.

When the dynamization mechanism 38 is in the unlocked mode, the biasing member 52 may be configured to oppose axial collapse of the strut assembly 22 under load. In particular, the biasing member 52 may resist axial movement of the strut body 30 relative to the dynamization shaft 40 and may store energy during collapse of the strut assembly 22. Upon reduction or removal of the applied load, the biasing member 52 may urge the strut body 30 to move in an opposite axial direction along the strut axis SX, thereby returning the strut assembly 22 toward its original or preset length.

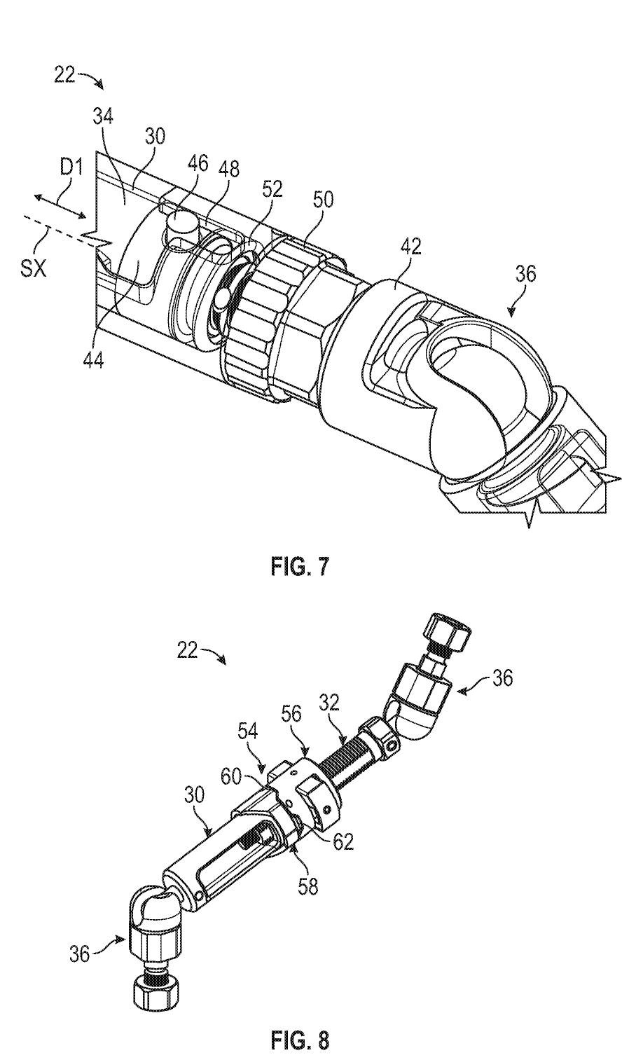

The retention pin 46 may contact a distal end of the retention slot 48 to limit relative axial movement between the strut body 30 and the dynamization shaft 40. In the locked mode, the strut body 30 may be axially constrained between the retention pin 46 and the dynamization adjustment control 50, as illustrated in FIG. 6A. In the unlocked mode, the strut body 30 may be movable between a first axial boundary established by the retention pin 46, as shown in FIG. 6B, and a second axial boundary established by the dynamization adjustment control 50, as shown in FIG. 7. As illustrated in FIG. 7, the strut body 30 may be configured to abut the dynamization adjustment control 50 to limit further movement of the strut body 30 in the direction D1. In this condition, the retention pin 46 may be spaced apart from the adjacent end of the retention slot 48 while the strut assembly 22 is in a collapsed state.

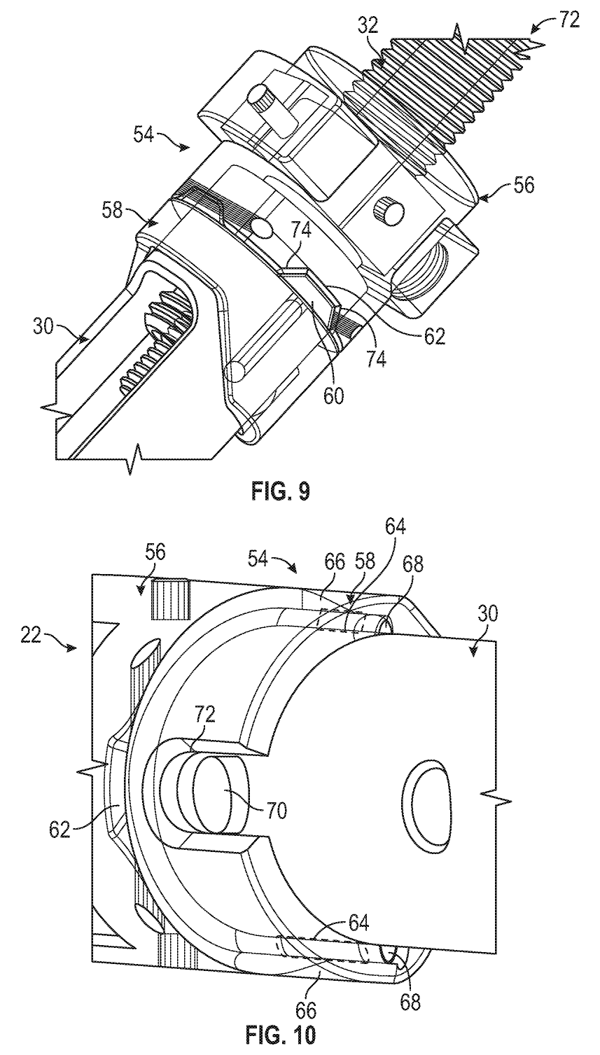

FIGS. 8-10 illustrate an exemplary incremental tactile feedback mechanism 54 incorporated into the strut assembly 22. The incremental tactile feedback mechanism 54 may be configured to enable incremental adjustment of an axial length of the strut assembly 22, for example by axially translating the threaded rod assembly 32 relative to the strut body 30. The incremental tactile feedback mechanism 54 may be further configured to generate perceptible tactile and/or audible feedback during adjustment, thereby facilitating accurate, repeatable modification of the strut length to achieve a prescribed postoperative deformity correction rate.

The incremental tactile feedback mechanism 54 may include a quick‑adjust knob 56 and a spring collar 58. The quick‑adjust knob 56 may be operably coupled to the threaded rod assembly 32 and may be rotatable relative to the strut body 30. The spring collar 58 may be movably mounted at an end portion of the strut body 30 adjacent the quick‑adjust knob 56.

The quick‑adjust knob 56 and the spring collar 58 may be configured to engage one another at one or more ramped engagement interfaces. For example, the spring collar 58 may include a plurality of ramp tabs 60 configured to selectively engage corresponding ramp slots 62 formed in the quick‑adjust knob 56. The ramped engagement interfaces may be circumferentially spaced to define discrete angular adjustment increments. Although ramp tabs and ramp slots are illustrated and described, other ramped or cam‑based engagement configurations may be employed without departing from the scope of this disclosure.

The spring collar 58 may be resiliently biased toward the quick‑adjust knob 56 by one or more springs 64. The springs 64 may be received within grooves 66 formed on an interior surface of the spring collar 58 and may be configured to urge the spring collar 58 into an engaged position relative to the quick‑adjust knob 56 under normal conditions when the strut assembly 22 is not being adjusted.

The spring collar 58 may be configured to translate axially relative to the strut body 30 against the biasing force of the springs 64. In an exemplary implementation, a ball bearing 68 may be disposed within each groove 66 and may cooperate with corresponding features of the strut body 30 to guide controlled axial movement of the spring collar 58 along the strut body 30 and in a direction away from the quick-adjust knob 56. Although ball bearings 68 are illustrated and described, alternative guiding or bearing elements could alternatively be utilized.

The spring collar 58 may further be constrained against relative rotation with respect to the strut body 30. For example, an anti‑rotation pin 70 may be fixed to the strut body 30 and received within a corresponding axially extending slot 72 formed in the spring collar 58. This pin‑and‑slot arrangement may permit axial translation of the spring collar 58 while preventing rotational movement, thereby ensuring consistent interaction between the ramp tabs 60 and the ramp slots 62.

In operation, rotation of the quick‑adjust knob 56 about the strut axis SX relative to the spring collar 58 may effectuate incremental axial length adjustments of the strut assembly 22. As the quick‑adjust knob 56 is rotated, for example in a clockwise direction to increase axial length or in a counterclockwise direction to decrease axial length, an angled wall 74 of one of the ramp slots 62 may engage a corresponding ramp tab 60. Continued rotation may cause the ramp tab 60 to ride along the angled wall 74, thereby compressing the springs 64 and urging the spring collar 58 to translate axially away from the quick‑adjust knob 56. Upon completion of the rotational increment, the springs 64 may return the spring collar 58 to a subsequent engaged position in which the ramp tabs 60 seat within adjacent ramp slots 62 corresponding to a next discrete adjustment increment.

In this manner, the incremental tactile feedback mechanism 54 may produce a distinct tactile sensation and, in some cases, an audible indication at each discrete adjustment increment. This feedback may allow a surgeon or patient to reliably count adjustment increments and to perform precise intraoperative or postoperative strut adjustments in accordance with a prescribed treatment protocol. The incremental tactile feedback mechanism 54 may thereby reduce the likelihood of over‑adjustment or under‑adjustment and enhance overall usability and accuracy of the strut assembly 22.

In some implementations, each feedback event generated by the incremental tactile feedback mechanism 54 may correspond to a predetermined angular increment of rotation of the quick‑adjust knob 56. By way of example and not limitation, each feedback event may correspond to approximately one‑quarter turn of the quick‑adjust knob 56, with each incremental turn producing a corresponding incremental change in the axial length of the strut assembly 22, such as approximately 0.25 millimeters, for example. However, other angular increments and corresponding axial displacement values are likewise contemplated within the scope of this disclosure.

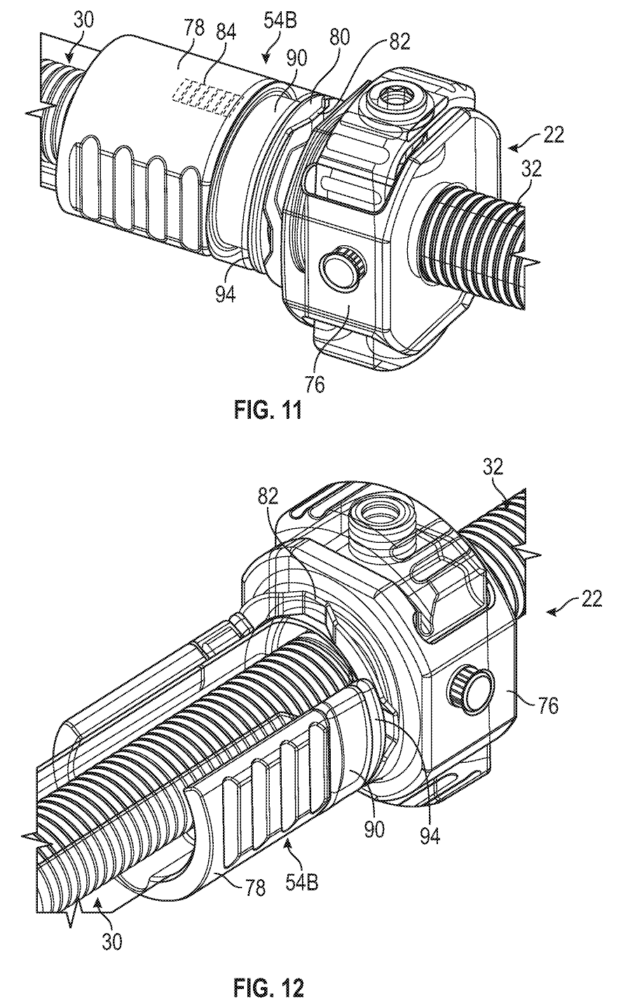

FIGS. 11-13 illustrate another exemplary incremental tactile feedback mechanism 54B that can be incorporated into the strut assembly 22. In this embodiment, the incremental tactile feedback mechanism 54B is configured as a cam-follower arrangement. The incremental tactile feedback mechanism 54B may operate on a similar quarter-turn incremental adjustment principle as that described above, while utilizing an alternative configuration to generate tactile and/or audible feedback during strut length adjustment.

The incremental tactile feedback mechanism 54B may include a quick-adjust housing 76 that functions as a cam and a cam adjustment collar 78 that functions as a follower. The cam adjustment collar 78 may be positioned adjacent an end portion of the strut body 30 and may be operably associated with the quick-adjust housing 76 such that relative rotation between these components results in incremental axial translation corresponding to axial adjustment of the threaded rod assembly 32.

The cam adjustment collar 78 may include at least one ramped engagement surface configured to interact with one or more corresponding cam features formed on the quick-adjust housing 76. In an exemplary implementation, the cam adjustment collar 78 may include a single ramp tab 80, with the ramp tab 80 engaging a series of cam ramps 82 circumferentially spaced about the quick-adjust housing 76 to define discrete adjustment increments.

The cam adjustment collar 78 may be resiliently biased toward the quick-adjust housing 76 by a single spring 84 such that, during rotation of the quick-adjust housing 76, the ramp tab 80 is sequentially urged over adjacent cam ramps 82. As the ramp tab 80 transitions between the cam ramps 82, the spring 84 may be temporarily compressed and then allowed to re-expand, thereby producing a perceptible tactile sensation and, in some implementations, an audible indication corresponding to each discrete adjustment increment.

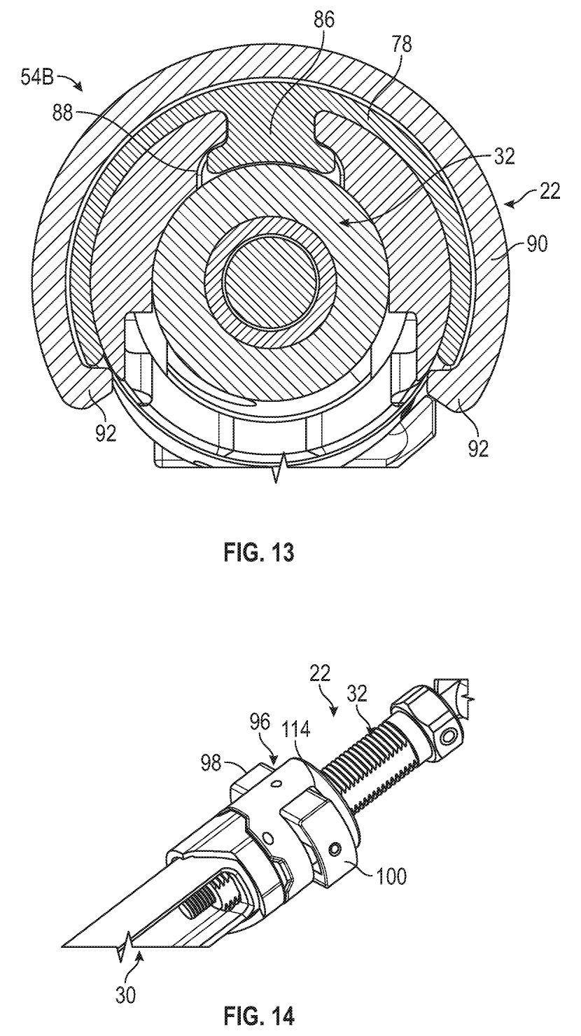

The cam adjustment collar 78 may further include a guide feature configured to prevent relative rotation with respect to the strut body 30 while permitting axial translation. For example, a T-shaped projection 86 formed on the cam adjustment collar 78 may be received within a corresponding slot 88 formed in an end of the strut body 30 (see FIG. 13), thereby constraining the cam adjustment collar 78 against rotation and ensuring consistent cam-follower interaction during adjustment.

In this manner, the incremental tactile feedback mechanism 54B may provide reliable, repeatable incremental feedback while employing fewer components and a simplified assembly. The cam-follower configuration may enable users to accurately count adjustment increments and perform precise intraoperative or postoperative adjustments of the strut assembly 22, while maintaining the tactile clarity and adjustment accuracy described herein.

The incremental tactile feedback mechanism 54B may further include a strut identification clip 90 configured to be removably coupled to the cam adjustment collar 78. The strut identification clip 90 may be configured to designate a strut identification number, such as for use in hexapod deformity correction configurations, for example. The strut identification clip 90 may clip over an exterior surface of the cam adjustment collar 78, such as defined at a cutout section 94 of the cam adjustment collar 78, and may include one or more clip tabs 92 configured to releasably engage corresponding features of the cam adjustment collar 78. In some implementations, the strut identification clip 90 may be omitted from the strut assembly 22, such as where the strut assembly 22 is used in a static reduction configuration that does not require strut number identification.

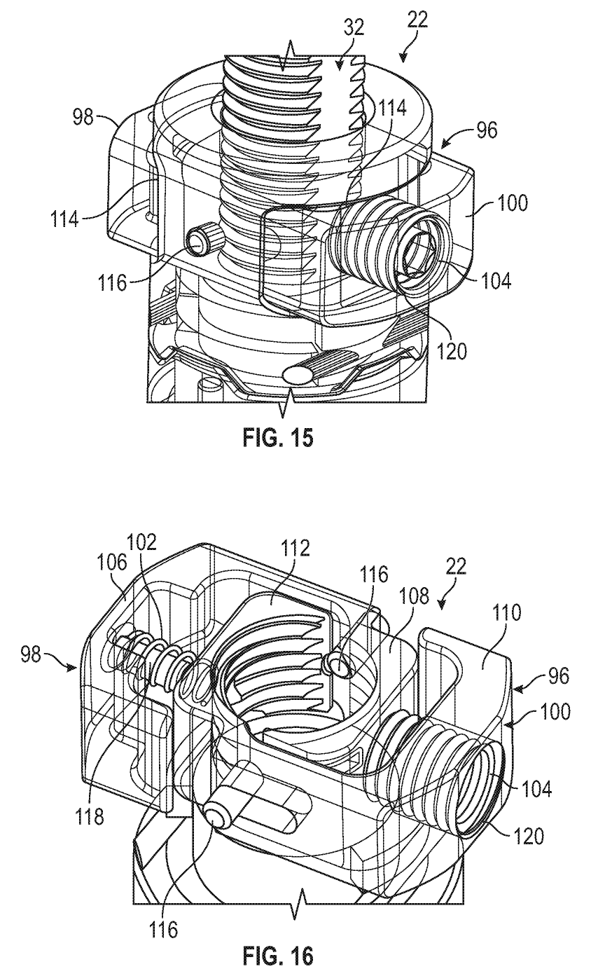

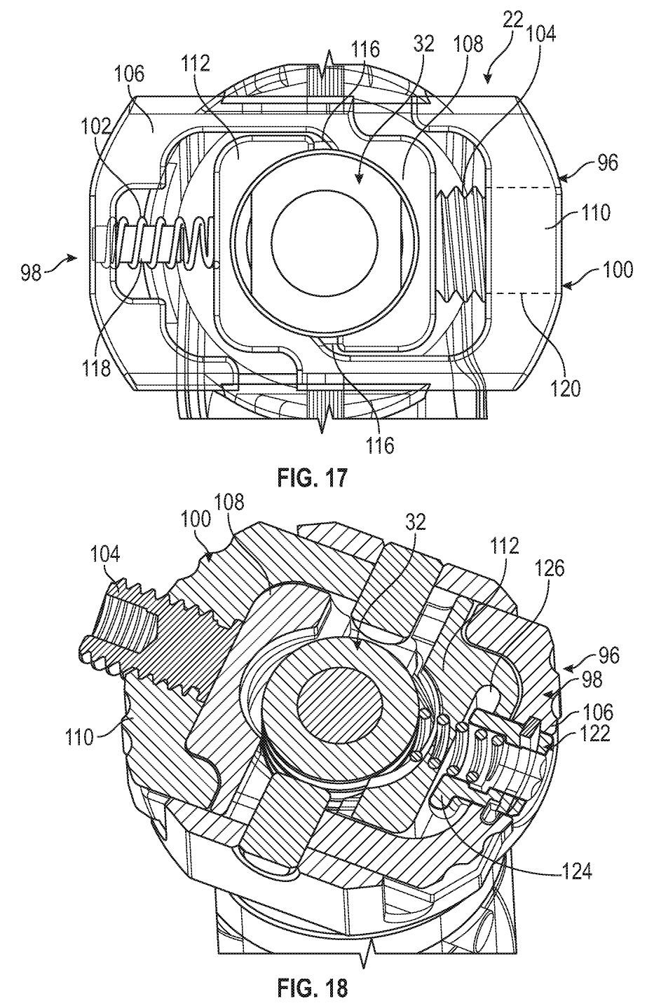

FIGS. 14–17 illustrate an exemplary macro quick adjust mechanism 96 incorporated into the strut assembly 22. The macro quick adjust mechanism 96 may be configured to permit rapid, tool‑free telescopic macro adjustment of an axial length of the strut assembly 22, such as during intraoperative setup or macro postoperative strut length changes.

The macro quick adjust mechanism 96 may include a first quick adjust button 98, a second quick adjust button 100, a spring 102, and a set screw 104. The spring 102 may be disposed between the first and second quick adjust buttons 98, 100 to bias the buttons toward an engaged configuration. The set screw 104 may be removably positioned between the first and second quick adjust buttons 98, 100 at a location diametrically opposed to the spring 102, and may be configured to selectively restrict or permit operation of the macro quick adjust mechanism 96.

The first quick adjust button 98 may include a first actuatable button portion 106 and a first thread‑engagement portion 108, and the second quick adjust button 100 may include a second actuatable button portion 110 and a second thread‑engagement portion 112. The first actuatable button portion 106 and the first thread‑engagement portion 108 may be integrally formed as a unitary body, and the second actuatable button portion 110 and the second thread‑engagement portion 112 may likewise be integrally formed as a unitary body. The first and second thread‑engagement portions 108, 112 may be configured to selectively engage corresponding threads of the threaded rod assembly 32.

The first quick adjust button 98 and the second quick adjust button 100 may be operably coupled to one another such that they function as a single actuatable assembly. For example, two or more lock pins 116 may extend between the first and second quick adjust buttons 98, 100 to synchronize movement of the buttons between engaged and disengaged positions relative to the threaded rod assembly 32.

In an assembled state of the strut assembly 22, the first actuatable button portion 106 and the second actuatable button portion 110 may each protrude through a respective opening 114 formed in the quick‑adjust knob 56, thereby permitting user access. In an engaged state of the strut assembly 22, the first and second thread‑engagement portions 108, 112 may engage the threaded rod assembly 32 to inhibit relative axial movement between the threaded rod assembly 32 and the strut body 30.

The spring 102 may be arranged over a dedicated pin 118 coupled to the first actuatable button portion 106 of the first quick adjust button 98. The pin 118 may extend from the first actuatable button portion 106 toward the second quick adjust button 100, with the spring 102 received about the pin 118. Opposing ends of the spring 102 may bear against seating surfaces provided by the first actuatable button portion 106 of the first quick adjust button 98 and the second thread‑engagement portion 112 of the second quick adjust button 100. In this manner, the spring 102 may resiliently bias the first and second thread‑engagement portions 108, 112 toward an engaged state with the threaded rod assembly 32. The pin 118 may further function as a guide for the spring 102 while permitting coordinated inward movement of the first and second quick adjust buttons 98, 100 during user actuation.

The set screw 104 may be configured to selectively lock out operation of the macro quick adjust mechanism 96. In particular, advancement of the set screw 104 may restrict inward movement of the first and second quick adjust buttons 98, 100, thereby maintaining the first and second thread‑engagement portions 108, 112 in engagement with the threaded rod assembly 32 regardless of user input. Such a lockout configuration may be advantageous, for example, when multiple strut assemblies 22 are adjusted simultaneously or when inadvertent macro adjustment is undesirable. In some implementations, the set screw 104 may be received within a threaded opening 120 formed in the second actuatable button portion 110 of the second quick adjust button 100 and may bear against a seating surface provided by the first thread‑engagement portion 108 of the first quick adjust button 98 when fully inserted. Retraction of the set screw 104 away from the seating surface may permit normal operation of the macro quick adjust mechanism 96 by allowing the first and second quick adjust buttons 98, 100 to move inward toward one another against the biasing force of the spring 102.

In operation, the macro quick adjust mechanism 96 may be actuated to rapidly change the axial length of the strut assembly 22 without requiring incremental rotation of the threaded rod assembly 32. To perform such a macro length adjustment, a user may first disengage the set screw 104 to permit inward movement of the first and second actuatable button portions 106, 110. The user may then simultaneously press the first and second actuatable button portions 106, 110 inwardly toward one another. In response to this actuation, the first and second thread‑engagement portions 108, 112 may be urged radially outward and thereby disengage from the threaded rod assembly 32 against the bias of the spring 102. While disengaged, the threaded rod assembly 32 may be freely translated axially relative to the strut body 30, thereby allowing rapid telescopic extension or contraction of the strut assembly 22 to a desired axial length. Upon subsequent release of the first and second actuatable button portions 106, 110, the spring 102 may return the first and second quick adjust buttons 98, 100 toward the engaged position, causing the first and second thread‑engagement portions 108, 112 to re‑engage the threaded rod assembly 32 and secure the strut assembly 22 at the desired adjusted length.

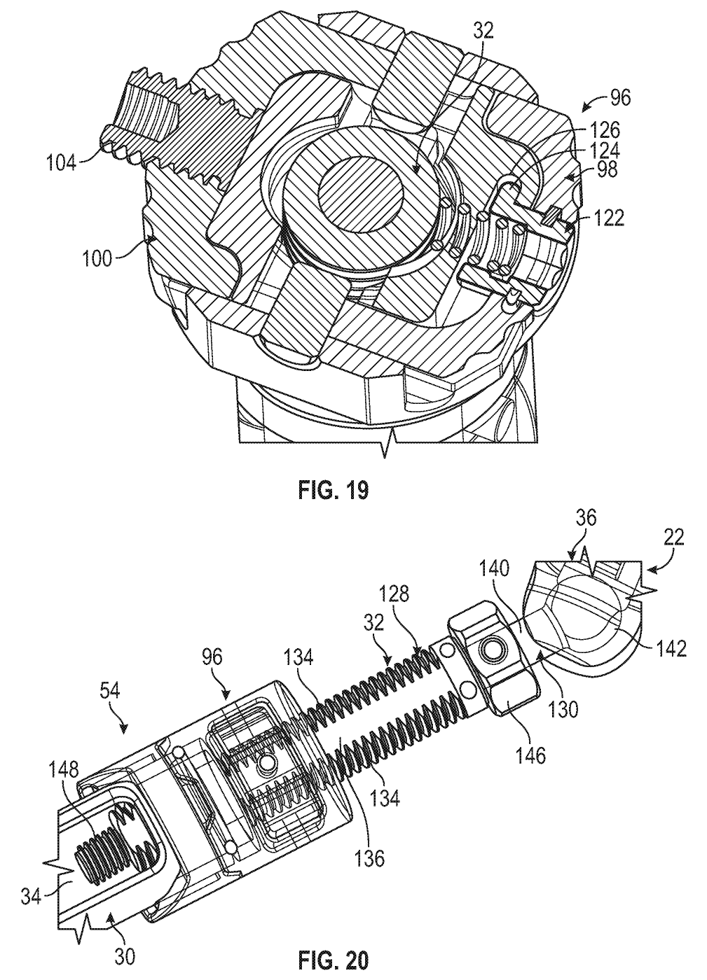

Referring to FIGS. 18-19, the macro quick adjust mechanism 96 may include, in some implementations, a lock knob 122 configured to selectively lock out operation of the macro quick adjust mechanism 96 so that macro adjustments can be made without the need to press the first and second quick adjust buttons 98, 100. The lock knob 122 may be rotatably coupled to one of the quick adjust buttons (here, the first quick adjust button 98) and may be moved between an unlocked position (see FIG. 18), in which the macro quick adjust mechanism 96 is operable, and a locked position (see FIG. 19), in which the first and second quick adjust buttons 98, 100 are locked in the compressed position relative to one another.

The lock knob 122 may be configured to engage corresponding structures of the macro quick adjust mechanism 96 when the lock knob 122 is rotated to the locked position. For example, rotation of the lock knob 122 to the locked position may cause a locking feature 124 to engage a keyway 126 of the second thread-engagement portion 112 of the second quick adjust button 100 and thereby hold the first and second quick adjust buttons 98, 100 in the compressed position in which the macro quick adjust mechanism 96 is disengaged from the threaded rod assembly 32.

In the unlocked position, the lock knob 122 may permit normal operation of the macro quick adjust mechanism 96, including inward actuation of the first and second quick adjust buttons 98, 100 to disengage the thread‑engagement portions 108, 112 from the threaded rod assembly 32. In the locked position, the lock knob 122 may maintain the macro quick adjust mechanism 96 in a secured configuration without requiring continuous user input, thereby allowing a user to perform simultaneous macro adjustments of multiple strut assemblies 22 more easily.

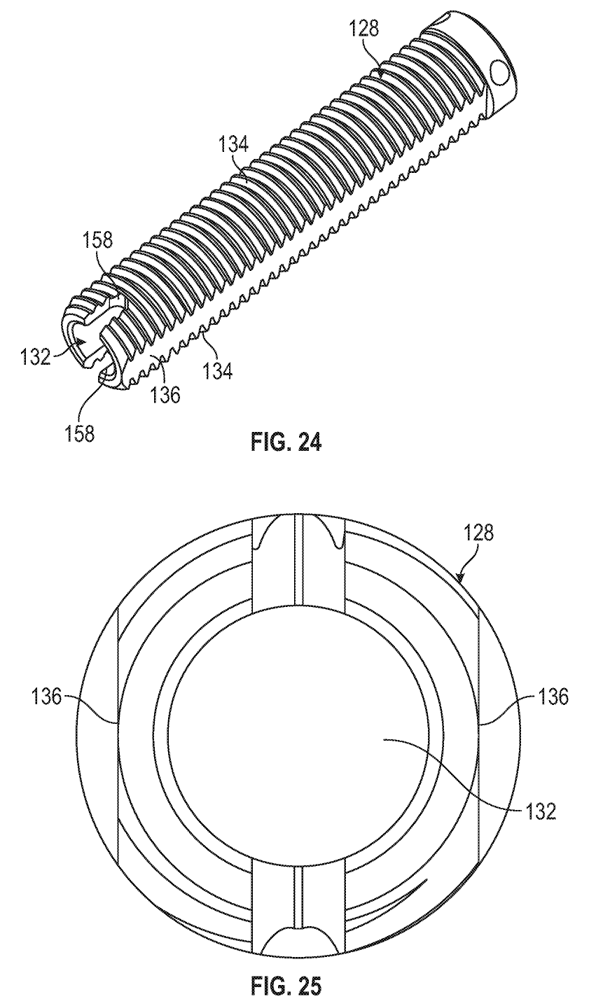

FIGS. 20–25 illustrate an exemplary threaded rod assembly 32 of the strut assembly 22. The threaded rod assembly 32 may include an outer extension tube 128 and an inner rod 130 received through a cannulation 132 formed through the outer extension tube 128. The inner rod 130 and the outer extension tube 128 may cooperate to provide controlled axial length adjustments of the strut assembly 22.

The outer extension tube 128 may be axially translatable relative to the strut body 30 while being constrained against relative rotation. The outer extension tube 128 may be operably coupled to the strut body 30 for controlled axial translation via the incremental tactile feedback mechanism 54 and/or the macro quick adjust mechanism 96 described above. In some implementations, the outer extension tube 128 may be constrained against rotational movement relative to the strut body 30 by the anti‑rotation pin 70. For example, the anti‑rotation pin 70 may engage a non‑circular surface or flat section 136 of the outer extension tube 128 to inhibit rotation while permitting axial movement.

The outer extension tube 128 may include a plurality of threaded sections 134 and a plurality of flat sections 136. In an embodiment, the threaded sections 134 and the flat sections 136 may extend along a majority, or substantially the entire axial length, of the outer extension tube 128. The threaded sections 134 and the flat sections 136 may be circumferentially arranged in alternating fashion about the outer extension tube 128.

The threaded sections 134 may each include external threads configured to be selectively engaged and disengaged by the first and second thread‑engagement portions 108, 112 of the first and second quick adjust buttons 98, 100. Engagement of the threaded sections 134 by the thread‑engagement portions 108, 112 may inhibit axial movement of the threaded rod assembly 32 relative to the strut body 30, while disengagement may permit telescoping axial movement for length adjustments.

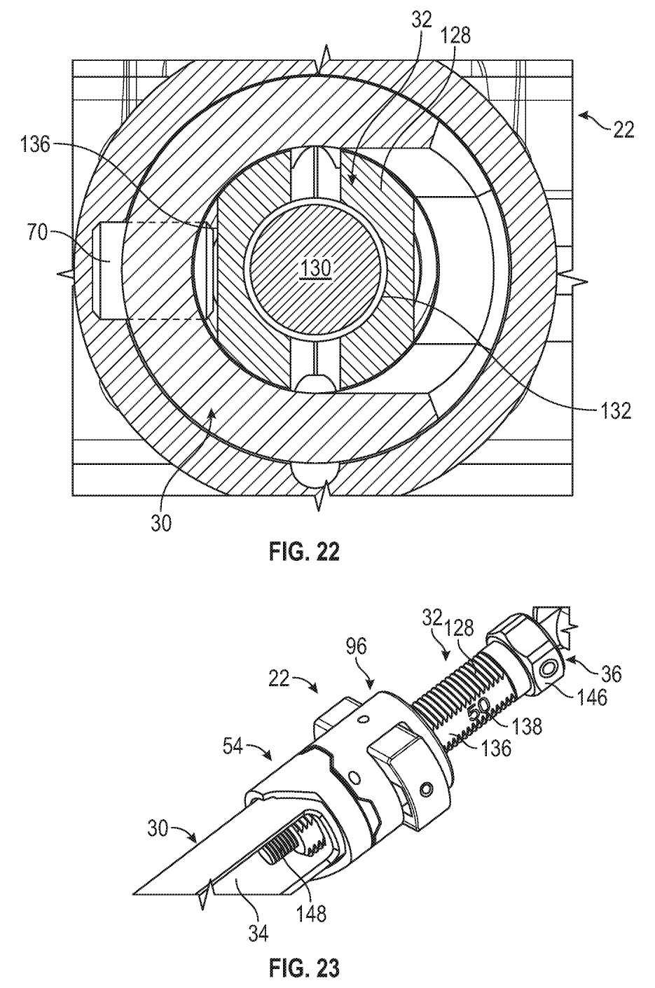

The flat sections 136 may be diametrically opposed from one another. One of the flat sections 136 may define a seating surface against which the anti‑rotation pin 70 bears to prevent rotational movement of the outer extension tube 128 relative to the strut body 30 (see FIG. 22). The other flat section 136 may include laser markings 138 (see FIG. 23) for visually conveying information (e.g., strut length or extension status) to a user during intraoperative setup and/or postoperative adjustment of the strut assembly 22. The laser markings 138 may include numeric indicators, symbols, orientation markers, or any combination thereof.

The inner rod 130 of the threaded rod assembly 32 may include a shaft portion 140 and a ball portion 142 integrally formed with the shaft portion 140. The shaft portion 140 may be received within the cannulation 132 of the outer extension tube 128, while the ball portion 142 may interface with the joint assembly 36 coupled to the threaded rod assembly 32 to permit angular articulation of the strut assembly 22.

The inner rod 130 may be rotationally movable relative to the outer extension tube 128 while being restrained against relative axial movement. For example, one or more coupling members 144, such as pins, may be carried by one of the inner rod 130 or the outer extension tube 128 and received within corresponding circumferential grooves, slots, or recesses formed in the other. The coupling members 144 may permit relative rotation between the inner rod 130 and the outer extension tube 128 while inhibiting axial translation therebetween. In this manner, the inner rod 130 may spin freely relative to the outer extension tube 128 while remaining axially retained, such as when the ball portion 142 is unlocked relative to the joint assembly 36.

A coupling fastener 146 may be mounted to the shaft portion 140 of the inner rod 130 at a location near a proximal end of the outer extension tube 128. The coupling fastener 146 may be a hex nut, although other rotatable coupling members capable of transmitting torque may likewise be utilized within the scope of this disclosure. A user may rotate the coupling fastener 146 in a clockwise or counterclockwise direction to impart rotational motion to the inner rod 130 relative to the outer extension tube 128.

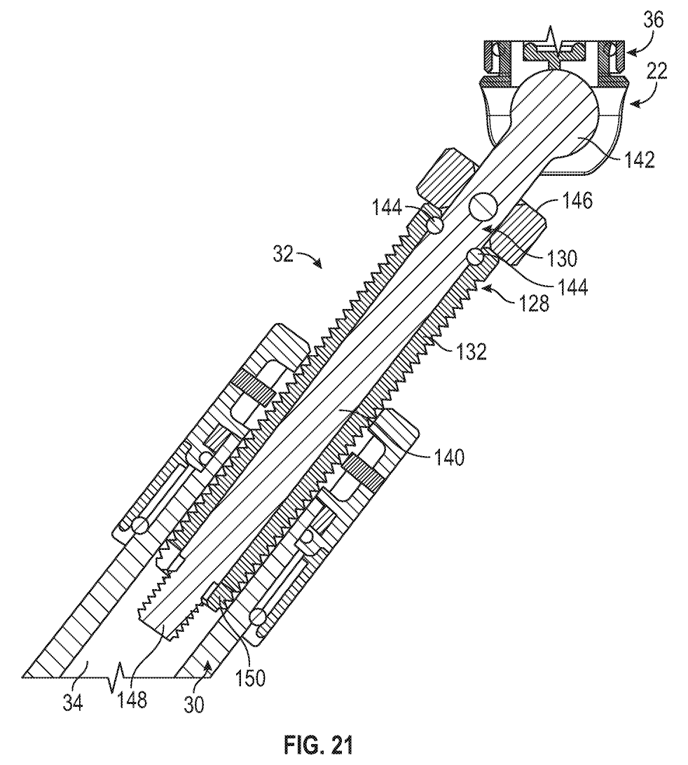

The inner rod 130 may further include a threaded end portion 148 located on an end opposite the ball portion 142. The threaded end portion 148 may project outwardly from a distal end of the outer extension tube 128 and may be at least partially received within the interior cavity 34 of the strut body 30. When the strut assembly 22 is configured for non‑hexapod applications, an extension cap 150 (see FIG. 21) may be secured to the threaded end portion 148 to lock the strut assembly 22 in all degrees of freedom.

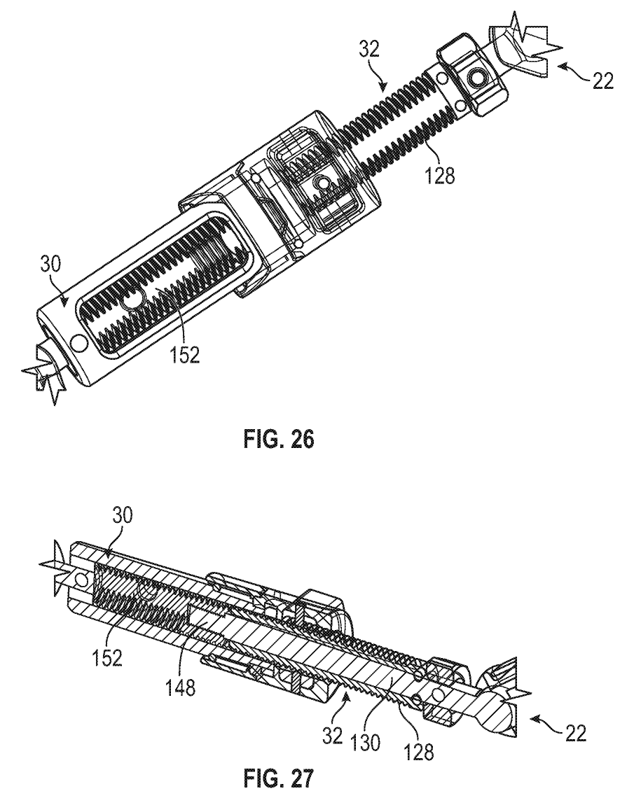

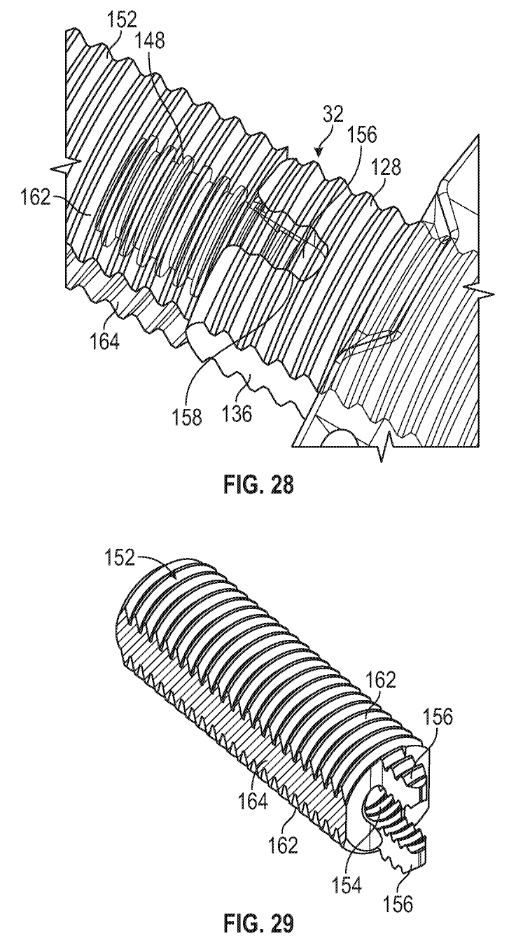

Referring to FIGS. 26–29, the threaded rod assembly 32 may further include an add‑on rod or secondary rod 152 configured to increase an overall available length of the threaded rod assembly 32, such as for accommodating larger patient anatomies or extended correction requirements, for example. The secondary rod 152 may be selectively secured to the threaded end portion 148 of the inner rod 130 to extend the effective length of the threaded rod assembly 32. In some implementations, the secondary rod 152 may include a threaded opening 154, such as a blind hole, configured to threadably receive the threaded end portion 148 for mechanically coupling the secondary rod 152 to the inner rod 130.

The secondary rod 152 and the outer extension tube 128 may include cooperating mating features for seamlessly connecting the secondary rod 152 to the threaded rod assembly 32. For example, in an exemplary implementation, the secondary rod 152 may include tangs 156 that can be received within corresponding slots 158 of the outer extension tube 128 for mating the secondary rod 152 and the outer extension tube 128 together. However, an opposite configuration is also contemplated, in which the outer extension tube 128 provides the tangs and the secondary rod 152 provides the slots.

When the secondary rod 152 is coupled to the threaded rod assembly 32, the threaded engagement between the threaded end portion 148 of the inner rod 130 and the threaded opening 154 of the secondary rod 152, together with the cooperating tang-in-slot mating features 156, 158, may establish a substantially rigid, load‑bearing connection that promotes alignment and reduces relative motion at the rod interface, thereby enhancing structural continuity and maintaining predictable load transfer during use. The combined structure thus functions mechanically as a single, elongated threaded rod assembly.

Similar to the outer extension tube 128, the secondary rod 152 may include a plurality of threaded sections 162 and a plurality of flat sections 164. The threaded sections 162 and the flat sections 164 may extend along a majority, or substantially the entire axial length, of the secondary rod 152. The threaded sections 162 and the flat sections 164 may be circumferentially arranged in alternating fashion about the secondary rod 152.

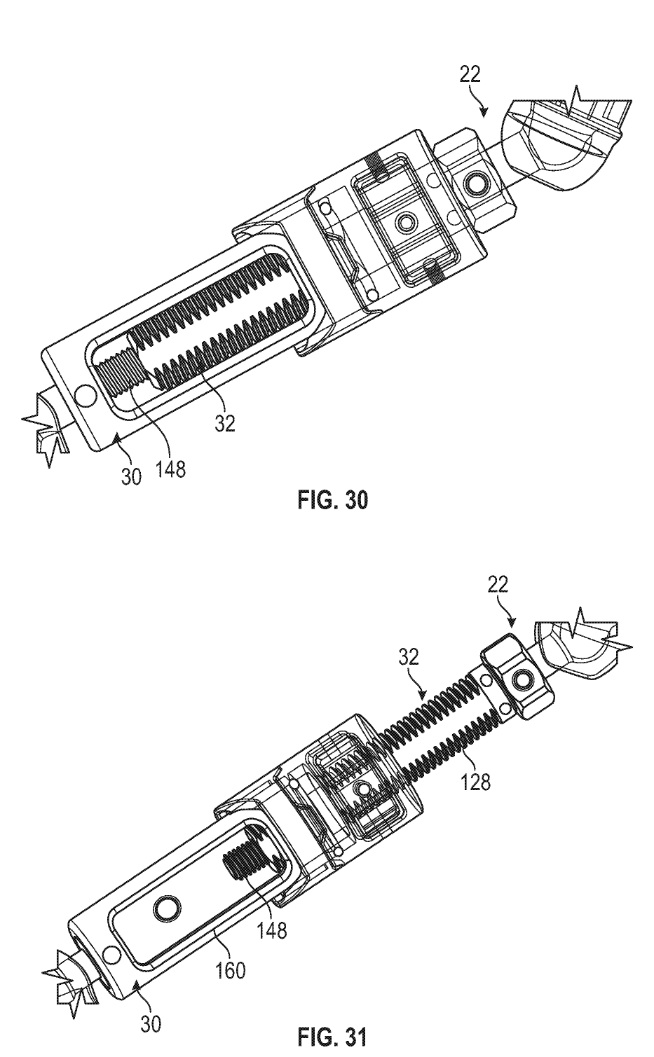

The secondary rod 152 may be selectively attachable to and detachable from the threaded rod assembly 32 to permit reversible configurations of the strut assembly 22. FIGS. 30–33 schematically illustrate an exemplary attachment method for securing the secondary rod 152 to the threaded rod assembly 32 to increase the available axial length of the strut assembly 22. The attachment method illustrated in FIGS. 30–33 may also be performed in reverse order to detach the secondary rod 152 from the threaded rod assembly 32.

The attachment method may begin with the strut assembly 22 in a fully contracted state (see FIG. 30). From the fully contracted state, the threaded rod assembly 32 may be extended to position the outer extension tube 128 relative to a loading slot 160 formed in the strut body 30, thereby providing access for insertion of the secondary rod 152 (see FIG. 31). In an exemplary implementation, the threaded rod assembly 32 may be transitioned to the extended configuration using the macro quick adjust mechanism 96, although other adjustment techniques are also contemplated.

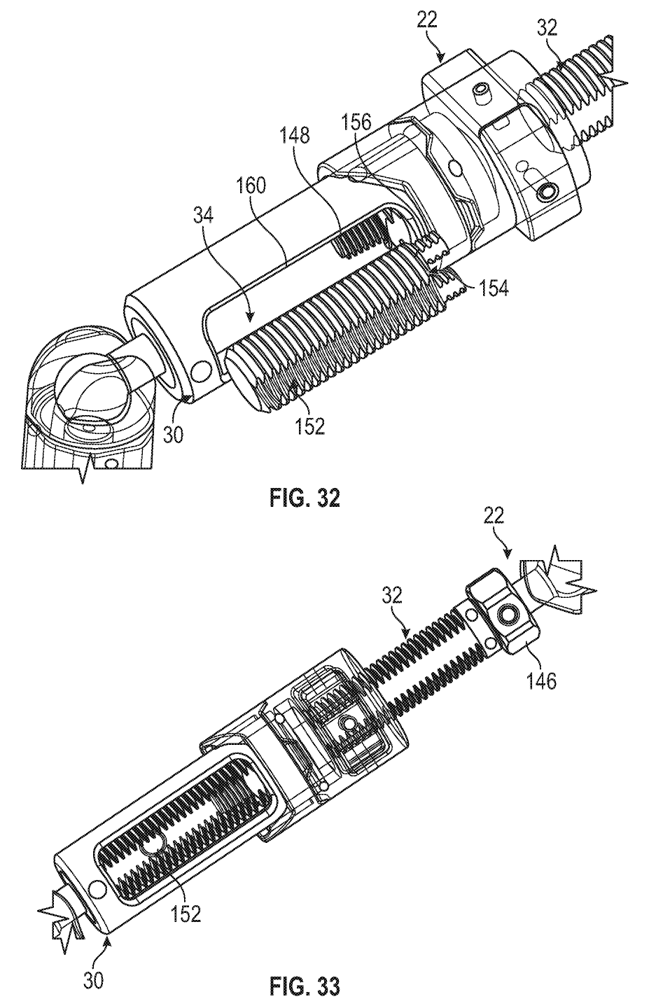

With the loading slot 160 exposed, the secondary rod 152 may be inserted laterally into the interior cavity 34 of the strut body 30 through the loading slot 160 (see FIG. 32). During insertion, the secondary rod 152 may be oriented such that one or more tangs 156 formed on the secondary rod 152 are aligned with corresponding slots 158 formed in the outer extension tube 128. Alignment of the tangs 156 with the slots 158 may facilitate proper rotational orientation of the secondary rod 152 relative to the threaded rod assembly 32.

The secondary rod 152 may then be positioned such that the threaded opening 154 of the secondary rod 152 is axially aligned with the threaded end portion 148 of the inner rod 130. The coupling fastener 146 may then be rotated, such as via a hex tool or other tool‑engaging feature, to threadably engage the threaded end portion 148 with the threaded opening 154 of the secondary rod 152 (see FIG. 33). Continued rotation of the coupling fastener 146 may draw the secondary rod 152 axially into engagement with the threaded rod assembly 32 until the mating features 156, 158 are fully seated and the connection is secured.

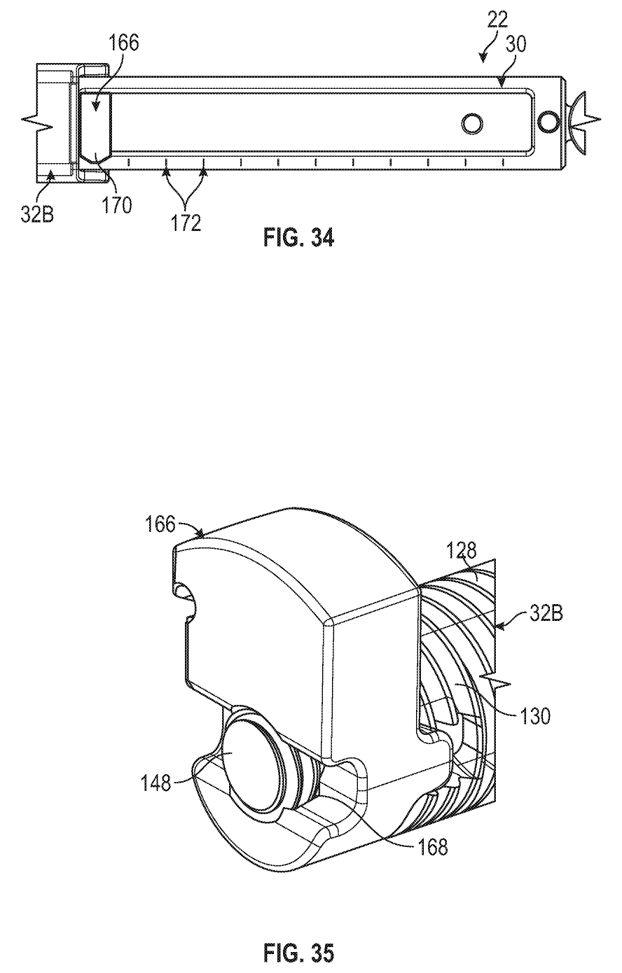

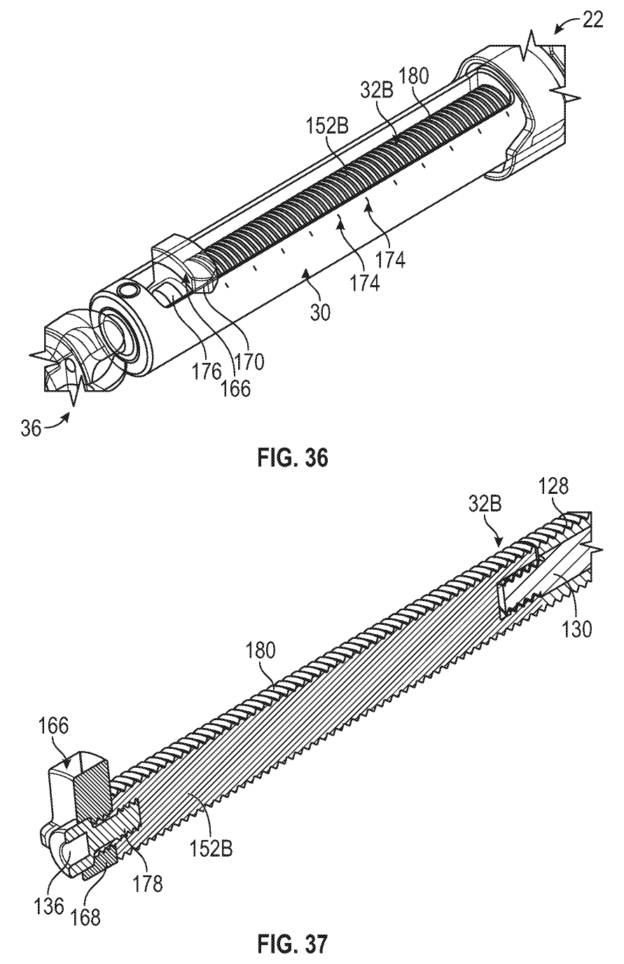

FIGS. 34–37 illustrate another exemplary threaded rod assembly 32B for use with the strut assembly 22. The threaded rod assembly 32B may include the outer extension tube 128, the inner rod 130, a modular indicator member 166, and, in some configurations, a secondary rod 152B. In this alternative embodiment, the modular indicator member 166 is configured to selectively cooperate with either the inner rod 130 (see FIGS. 34–35) or the secondary rod 152B (see FIGS. 36–37) to provide accurate strut length indication across multiple strut configurations.

The modular indicator member 166 may be selectively attachable to either the inner rod 130 or the secondary rod 152B, depending on whether the strut assembly 22 is configured in a base‑length configuration or an extended‑length configuration. The modular indicator member 166 may include a threaded opening 168 for establishing the connection to the inner rod 130 or the secondary rod 152B, and may further include an indicator pointer 170 configured to visually indicate strut length during adjustment of the threaded rod assembly 32B.

In the base‑length configuration of the threaded rod assembly 32B shown in FIGS. 34–35, in which the secondary rod 152B is not attached to the inner rod 130, the modular indicator member 166 may be secured directly to the inner rod 130. For example, the modular indicator member 166 may be threadably coupled to the threaded end portion 148 of the inner rod 130 by aligning the threaded opening 168 with the threaded end portion 148. In this configuration, the modular indicator member 166 may be oriented such that the indicator pointer 170 aligns with a first set of length designations 172 formed on the strut body 30, corresponding to a first length range of the threaded rod assembly 32B, thereby providing visual indication of strut length during base-length adjustment.

When it is desired to increase the available length of the strut assembly 22, the secondary rod 152B may be attached to the inner rod 130 in the manner described herein. Because the modular indicator member 166 occupies the threaded end portion 148 of the inner rod 130 when installed thereon, the modular indicator member 166 may be removed from the inner rod 130 prior to attaching the secondary rod 152B. Following attachment of the secondary rod 152B to the inner rod 130, the modular indicator member 166 may be flipped in orientation and releasably secured to the secondary rod 152B instead of the inner rod 130.

In an embodiment, the modular indicator member 166 may be secured to the secondary rod 152B by a fastener 176, such as a screw, for example. The fastener 176 may be inserted through the threaded opening 168 of the modular indicator member 166 and received within a corresponding threaded opening 178 formed in the secondary rod 152B to retain the modular indicator member 166 in the desired orientation.

In this implementation, the secondary rod 152B may include an outer diameter threading 180 and may omit flat sections or other anti‑rotation features. The outer diameter threading 180 may extend along a majority, or substantially an entire axial length, of the secondary rod 152B to permit threaded engagement and adjustment when installed.

In the extended‑length configuration of the threaded rod assembly 32B shown in FIGS. 36–37, the flipped orientation of the modular indicator member 166 may cause the indicator pointer 170 to align with a second, different set of length designations 174 formed on the strut body 30 corresponding to an extended length range of the threaded rod assembly 32B. In this manner, the modular indicator member 166 may provide accurate visual indication of strut length regardless of whether the secondary rod 152B is attached.

The ability to selectively relocate and reorient the modular indicator member 166 between the inner rod 130 and the secondary rod 152B may function as a configuration‑dependent reference mechanism that ensures the appropriate length scale is used for a given strut configuration. This arrangement may reduce ambiguity in strut length indication, minimize the likelihood of user error, and allow a single modular indicator member 166 to be used across multiple strut length ranges while preserving reversible attachment of the secondary rod 152B.

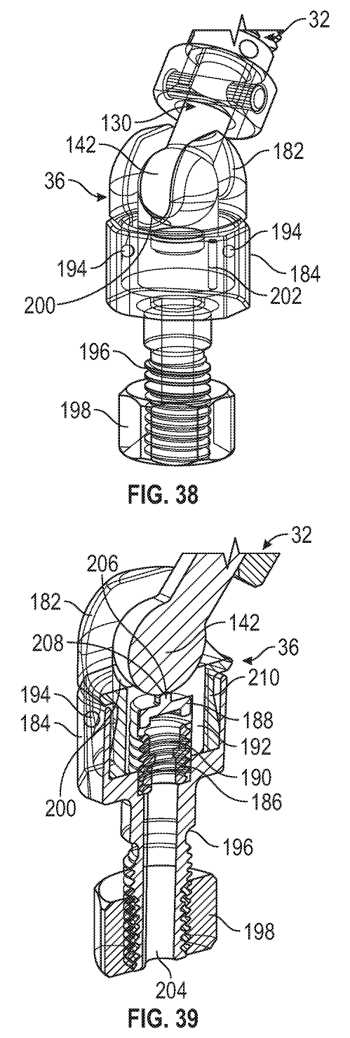

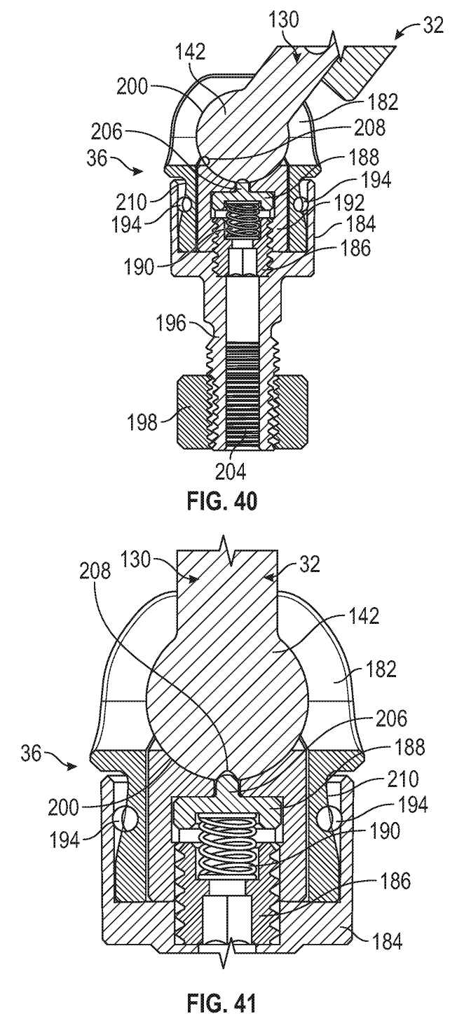

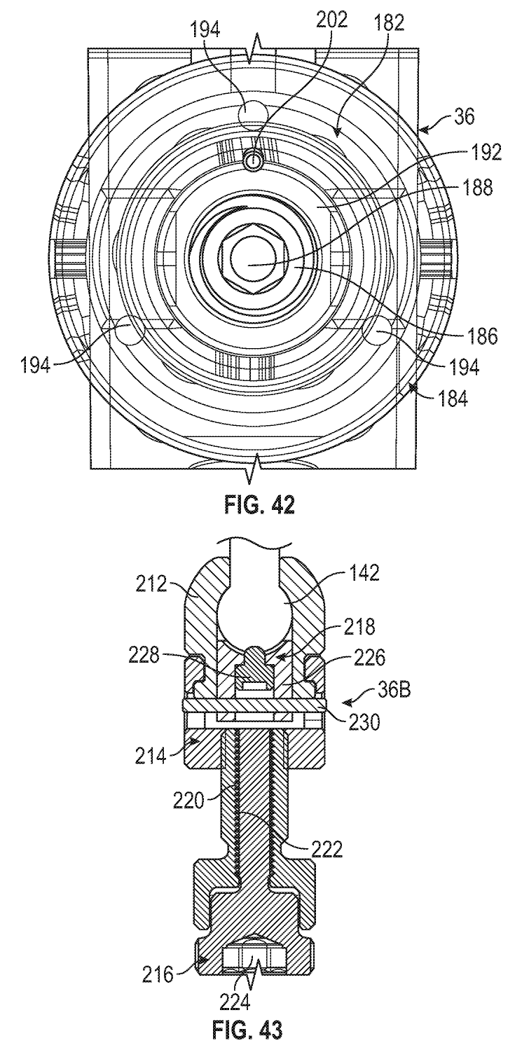

FIGS. 38–42 illustrate an exemplary joint assembly 36 for use with the strut assembly 22. In the illustrated embodiment, the joint assembly 36 is coupled to the threaded rod assembly 32 of the strut assembly 22 to facilitate attachment of the strut assembly 22 to the first support 24 or the second support 26 of the external fixation system 20. A corresponding joint assembly attached to the strut body 30 may include the same joint assembly design shown in FIGS. 38–42 or, alternatively, a different joint assembly design without departing from the scope of this disclosure.

The joint assembly 36 may include a swivel lock cap 182, a cannulated mount base 184, a threaded socket 192, a lock screw 186, a detent 188, and a biasing member 190. The swivel lock cap 182 may be configured to movably receive the ball portion 142 of the inner rod 130 of the threaded rod assembly 32, or another ball member operably connected to the strut assembly 22. When the joint assembly 36 is in an unlocked state, the ball portion 142 may swivel relative to the swivel lock cap 182, thereby permitting angular articulation of the strut assembly 22. In an embodiment, the ball portion 142 may swivel through a range of up to approximately 180 degrees relative to the swivel lock cap 182 when the joint assembly 36 is unlocked, although other ranges are also contemplated within the scope of this disclosure.

The cannulated mount base 184 may be rotatably coupled to the swivel lock cap 182. In an embodiment, the swivel lock cap 182 is rotatably supported relative to the cannulated mount base 184 by one or more rolling elements, such as ball bearings 194 (three shown), for example. The ball bearings 194 may be circumferentially spaced and positioned between the swivel lock cap 182 and the cannulated mount base 184 to facilitate smooth rotational movement while maintaining axial retention. Although ball bearings 194 are illustrated and described, other rotational support or bearing elements may be employed without departing from the scope of this disclosure. When the joint assembly 36 is in the unlocked state, the ball bearings 194 may permit the swivel lock cap 182 to rotate relative to the cannulated mount base 184 through a range of up to approximately 360 degrees.

The cannulated mount base 184 may include a shaft portion 196 extending away from the swivel lock cap 182. The shaft portion 196 may be received through an opening 31 formed in either the first support 24 or the second support 26 of the external fixation system 20. Once inserted through the opening 31, the shaft portion 196 may be secured in place by a retention member, such as a nut 198, thereby facilitating attachment of the strut assembly 22 to the first support 24 or the second support 26.

The threaded socket 192 may be movably positioned within an internal bore collectively defined by the swivel lock cap 182 and the cannulated mount base 184. The threaded socket 192 may include a curved seating surface 200 configured to receive and support the ball portion 142 of the inner rod 130 when the joint assembly 36 is assembled.

The lock screw 186 may be operably received within an internal bore provided by the threaded socket 192 and the cannulated mount base 184. The detent 188 may be operably positioned within an internal bore of the threaded socket 192. The biasing member 190, which may be a spring, may be positioned between opposing seating surfaces of the detent 188 and the lock screw 186 to selectively bias the detent 188 toward engagement with the ball portion 142 and to provide controlled resistance during adjustment of the joint assembly 36.

An anti-rotation pin 202 may be configured to prevent relative rotation between the threaded socket 192 and the swivel lock cap 182. In an embodiment, the anti-rotation pin 202 is fixed relative to the threaded socket 192 or the swivel lock cap 182 and received within a corresponding slot or recess formed in the other of the threaded socket 192 or the swivel lock cap 182. The anti-rotation pin 202 may inhibit rotational movement of the threaded socket 192 relative to the swivel lock cap 182 while maintaining the threaded socket 192 in a fixed axial position relative thereto. In this manner, rotation of the lock screw 186 may be translated into controlled axial advancement or retraction of the lock screw 186 relative to the threaded socket 192 without inducing rotation of the threaded socket 192 or the swivel lock cap 182, thereby preserving alignment of the ball portion 142 and ensuring consistent locking behavior of the joint assembly 36.

The joint assembly 36 may be selectively movable between a locked state and an unlocked state to control relative movement between the ball portion 142 and the swivel lock cap 182. The lock screw 186 may be operably coupled to internal threading of the threaded socket 192 such that rotation of the lock screw 186 causes axial movement of the lock screw 186 relative to the threaded socket 192. In particular, rotation of the lock screw 186 in a first direction may advance the lock screw 186 toward the swivel lock cap 182, while rotation in an opposite direction may retract the lock screw 186 away from the swivel lock cap 182. The lock screw 186 may be accessed for rotation via a cannulation 204 of the cannulated mount base 184.

When the lock screw 186 is advanced toward the detent 188, a detent pin 206 associated with the detent 188 may be urged into direct engagement with the ball portion 142. This engagement may generate a frictional and/or interference force that restricts relative movement of the ball portion 142 with respect to the swivel lock cap 182. In a fully locked state, the joint assembly 36 may substantially restrict or prevent angular movement between the ball portion 142 and the swivel lock cap 182, thereby fixing the angular orientation of the strut assembly 22 relative to the first support 24 or the second support 26 of the external fixation system 20.

The detent 188 and the biasing member 190 may cooperate to provide controlled resistance, tactile feedback, and positional stability during adjustment of the strut assembly 22. The detent 188 may be biased by the biasing member 190 toward selective engagement with the ball portion 142 or another component of the joint assembly 36. In some implementations, the detent 188 may cooperate with one or more recesses, depressions, or profiled features formed in the ball portion 142 to define one or more preferred angular positions. For example, the detent pin 206 may be received within a conical recess 208 of the ball portion 142 to assist in holding the strut assembly 22 at an orthogonal orientation relative to the first support 24 or the second support 26.

The biasing member 190 may apply a force vector that maintains engagement between the detent 188 and the ball portion 142 unless overcome by deliberate user‑applied force or by operation of the lock screw 186. This detent‑and‑biasing arrangement may assist in stabilizing the strut assembly 22 during angular positioning, inhibit unintended movement when the joint assembly 36 is unlocked or partially unlocked, and provide tactile feedback indicative of angular alignment or adjustment.

The swivel lock cap 182 may further include an inwardly tapered surface 210 configured to cooperate with the ball bearings 194 to selectively restrict rotation of the swivel lock cap 182 relative to the cannulated mount base 184. As the lock screw 186 is tightened, axial force may be transmitted through the threaded socket 192, causing the inwardly tapered surface 210 of the swivel lock cap 182 to advance toward the ball bearings 194. This movement may wedge the ball bearings 194 radially outward into firm engagement with corresponding bearing surfaces of the cannulated mount base 184, thereby increasing friction and inhibiting relative rotational movement between the swivel lock cap 182 and the cannulated mount base 184. In this manner, tightening of the lock screw 186 may simultaneously clamp the ball portion 142 and lock rotational movement of the swivel lock cap 182, while loosening the lock screw 186 may release the wedging action and permit smooth rotational adjustment.

The joint assembly 36 may further be configured to permit controlled post‑operative dynamization of the strut assembly 22. For example, partial retraction of the lock screw 186 from the fully locked position may reduce the clamping force applied to the ball portion 142 while maintaining sufficient contact to support axial loading. In such a partially unlocked condition, the joint assembly 36 may permit limited relative movement between the ball portion 142 and the cannulated mount base 184 in response to physiological loading.

The degree of dynamization may be adjusted by selectively rotating the lock screw 186 to vary the amount of clearance or compliance within the joint assembly 36. In some implementations, loosening the lock screw 186 beyond a predetermined threshold may introduce a defined gap or compliant zone that allows controlled axial or angular movement while still maintaining overall construct stability. This adjustable dynamization capability may allow a user to post-operatively tailor frame stiffness to promote bone healing, reduce stress shielding, and accommodate changes in patient loading over time.

In this manner, the joint assembly 36 may provide a unified mechanism for angular adjustment, rigid fixation, and selectively controlled dynamization without requiring removal or replacement of the strut assembly 22. The lock screw 186, detent 188, and biasing member 190 may cooperate to provide intuitive user control, repeatable adjustment, and reliable retention of the selected locking or dynamization state throughout the treatment period.

FIG. 43 illustrates another exemplary joint assembly 36B for use with the strut assembly 22. The joint assembly 36B may include a swivel lock cap 212, a cannulated mount base 214, a finger screw 216, and a detent assembly 218. This embodiment may provide a simplified strut‑to‑ring mount configuration suitable for manual or tool‑assisted locking and adjustment.

The cannulated mount base 214 may include an internally threaded receptacle 220 configured to threadably receive a corresponding external threading 222 of the finger screw 216. The finger screw 216 may be inserted through the cannulated mount base 214 and threadably engaged with the internally threaded receptacle 220. The finger screw 216 may further include a driver feature 224, such as a hex tool‑engaging feature, to permit tightening or loosening using a tool, while also allowing manual actuation if desired.

The detent assembly 218 may include a socket 226 and a detent pin 228 received within an internal bore of the socket 226. The detent assembly 218 may be movably positioned within an internal bore collectively defined by the swivel lock cap 212 and the cannulated mount base 214. A cross pin 230 may be provided to inhibit relative rotation between the detent assembly 218 and the cannulated mount base 214. In this manner, the cross pin 230 may maintain angular alignment of the detent assembly 218 while permitting axial movement thereof, such that rotation of the finger screw 216 results in controlled axial translation of the detent assembly 218 without inducing rotation relative to the swivel lock cap 212.

Rotation of the finger screw 216 in a first direction may advance the detent assembly 218, thereby urging the detent pin 228 into engagement with the ball portion 142 and restricting relative angular movement between the ball portion 142 and the swivel lock cap 212. Rotation of the finger screw 216 in an opposite direction may retract the detent assembly 218 away from the ball portion 142, thereby reducing clamping force and permitting angular adjustment of the strut assembly 22 relative to the external fixation system 20.

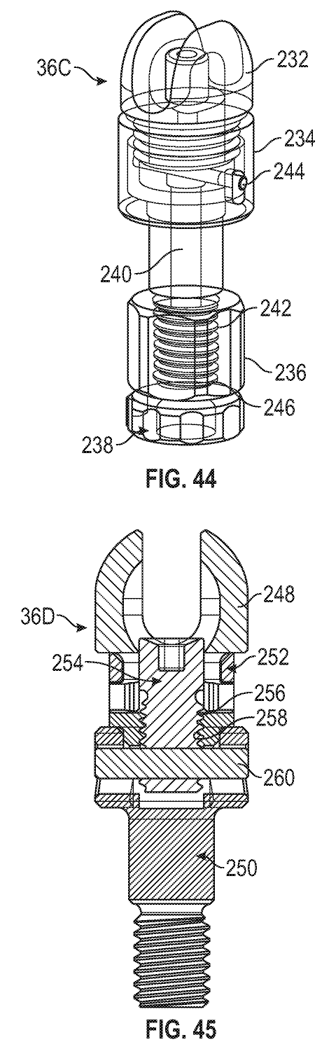

FIG. 44 illustrates another exemplary joint assembly 36C for use with the strut assembly 22. The joint assembly 36C may include a swivel lock cap 232, a cannulated mount base 234, a lock nut 236, a thumb screw 238, and a base post 240. This embodiment may provide an alternative strut‑to‑platform mount configuration that enables manual or tool‑assisted locking and angular adjustment of the strut assembly 22 relative to the external fixation system 20.

The cannulated mount base 234 may include external threads 242 configured to receive the lock nut 236 on a side of the joint assembly 36C opposite the swivel lock cap 232. The base post 240 may extend through the cannulated mount base 234 and may be axially movable relative thereto. A cross pin 244 may be coupled to the base post 240 to inhibit relative rotation between the base post 240 and the cannulated mount base 234 while permitting axial translation of the base post 240.

The thumb screw 238 may be threadably received within an internally threaded portion 246 of the lock nut 236 and may be configured to bear against the base post 240. Rotation of the thumb screw 238 in a first direction may advance the base post 240 toward a ball portion received by the swivel lock cap 232, thereby increasing clamping force and restricting relative angular movement between the ball portion and the swivel lock cap 232. Rotation of the thumb screw 238 in an opposite direction may retract the base post 240, thereby reducing clamping force and permitting relative angular movement between the ball portion and the swivel lock cap 232.

In this manner, the strut‑to‑platform mount configuration of FIG. 44 may provide a compact attachment mechanism in which axial clamping force is generated by actuation of the thumb screw 238, while the lock nut 236 secures the cannulated mount base 234 relative to the external fixation system platforms. This arrangement may allow intuitive adjustment, secure fixation, and reliable angular positioning of the strut assembly 22 during use.

FIG. 45 illustrates another exemplary joint assembly 36D for use with the strut assembly 22. The joint assembly 36D may include a swivel lock cap 248, a cannulated mount base 250, a lock body 252, and a threaded ball interface 254 configured to selectively lock or release angular movement of the strut assembly 22 relative to a fixation platform.

The threaded ball interface 254 may be operably coupled to the swivel lock cap 248 and the cannulated mount base 250. The threaded ball interface 254 may include external threads 256 configured to engage corresponding internal threads 258 of the lock body 252. A cross pin 260 may couple the threaded ball interface 254 to the cannulated mount base 250 such that the threaded ball interface 254 is constrained against relative rotation while permitting axial movement with respect to the swivel lock cap 248 and the cannulated mount base 250.

The lock body 252 may be rotationally connected to the swivel lock cap 248 and the cannulated mount base 250. Rotation of the lock body 252 in a first direction may cause the threaded ball interface 254 to be drawn axially into engagement with a ball portion received by the swivel lock cap 248, thereby clamping the ball portion and restricting relative angular movement. Rotation of the lock body 252 in an opposite direction may permit the threaded ball interface 254 to move axially away from the ball portion, thereby reducing clamping force and allowing relative angular movement between the ball portion and the swivel lock cap 248.

In this manner, the strut‑to‑platform mount configuration of FIG. 45 may provide a compact, coaxial locking mechanism in which rotational actuation of the lock body 252 is converted into axial clamping of the ball interface. This arrangement may enable secure fixation, controlled adjustment, and optional post‑operative dynamization without requiring disassembly of the strut assembly 22 from the external fixation system 20.

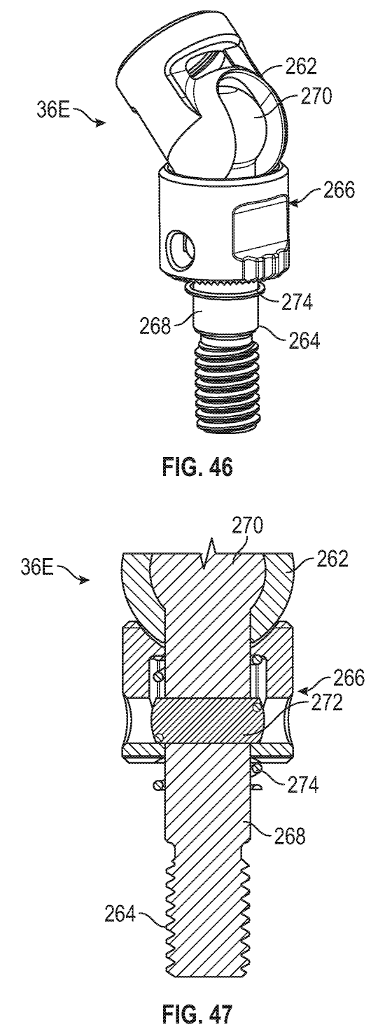

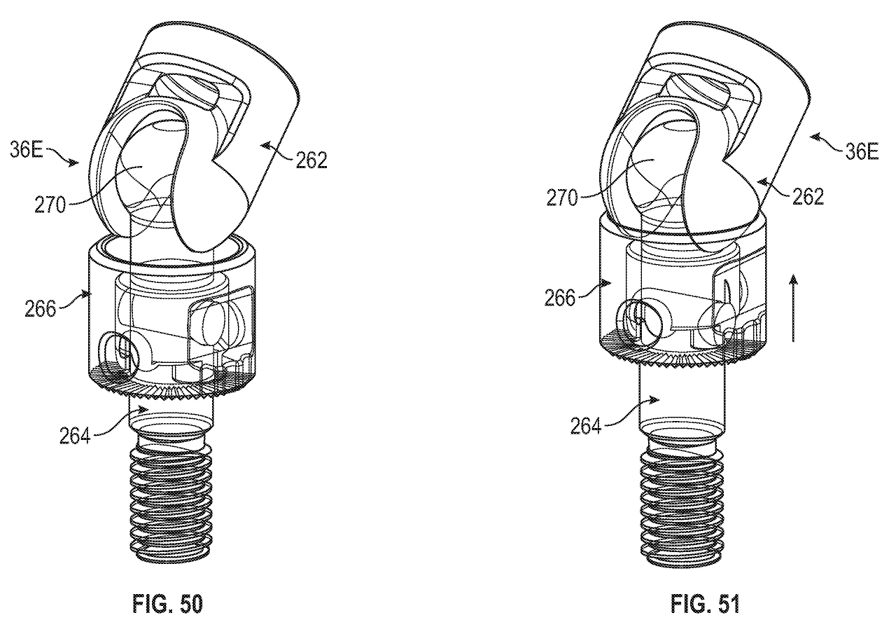

FIGS. 46–53 illustrate another exemplary joint assembly 36E for use with the strut assembly 22. The joint assembly 36E may provide a convertible configuration that is selectively operable in a first mode permitting angular articulation of the strut assembly 22 relative to the external fixation system 20 and in a second mode in which angular movement is restricted. In this manner, the joint assembly 36E may enable use of the strut assembly 22 in both a hexapod mode and a reduction mode.

The joint assembly 36E may include a swivel housing 262, a mounting rod 264, and a strut base 266. The mounting rod 264 may include a shaft portion 268 and a ball portion 270. The shaft portion 268 may be received through an opening 31 formed in either the first support 24 or the second support 26 of the external fixation system 20 and may be secured in place by a fastening element, such as a nut, to attach the strut assembly 22 to the external fixation system 20. The ball portion 270 may be received within and cooperate with the swivel housing 262 to permit angular articulation of the strut assembly 22.

The strut base 266 may be operably coupled to both the swivel housing 262 and the mounting rod 264. In an embodiment, the strut base 266 is coupled to the shaft portion 268 of the mounting rod 264 by a cross pin 272. An internal biasing member 274, such as a spring, may be disposed about the shaft portion 268 and positioned between the strut base 266 and the swivel housing 262. The internal biasing member 274 may be configured to bias the strut base 266 toward a selected operating condition relative to the swivel housing 262.

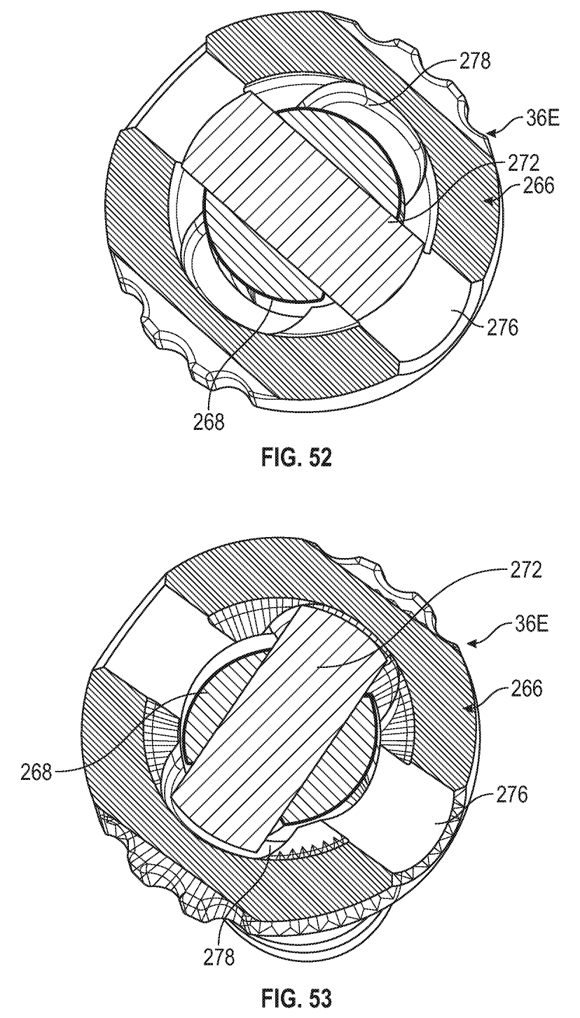

In a first, hexapod mode of the joint assembly 36E, the strut base 266 may be positioned in a first rotational orientation relative to the swivel housing 262. In this orientation, the cross pin 272 may be received within a first relief region 276 of the strut base 266 (see FIG. 52). The first relief region 276 may permit axial translation of the ball portion 270 relative to the strut base 266 as locking hardware is tightened. As a result, tightening the locking hardware may secure the strut assembly 22 to the external fixation system 20 while allowing the ball portion 270 to remain angularly articulated, thereby enabling dynamic hexapod operation.

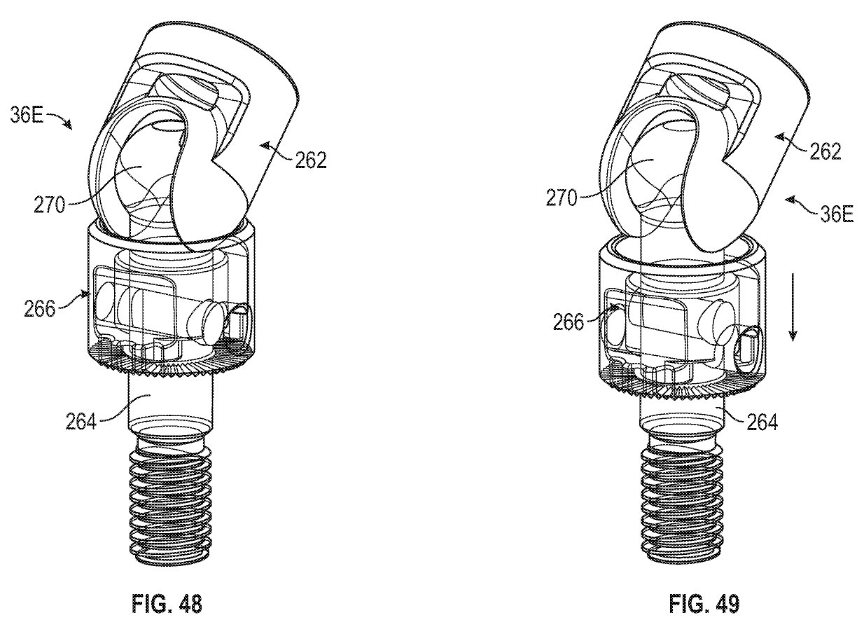

In a second, reduction mode of the joint assembly 36E, the strut base 266 may be repositioned relative to the swivel housing 262. For example, the strut base 266 may be pulled axially away from the swivel housing 262 against the biasing force of the internal biasing member 274 and then rotated to a second rotational orientation. In this orientation, the cross pin 272 may be received within a second, deeper relief region 278 of the strut base 266 (see FIG. 53). Upon tightening of the locking hardware, the altered geometry may cause the ball portion 270 to be drawn into firm engagement with the swivel housing 262, thereby restricting angular movement and placing the strut assembly 22 in a rigid, static reduction configuration. An exemplary sequence for converting the joint assembly 36E from the hexapod mode to the reduction mode is schematically illustrated in FIGS. 48–51.

The internal biasing member 274 may function to maintain the strut base 266 in the selected rotational orientation during use, while permitting intentional repositioning by a user without the need for tools or additional components. In this manner, the convertible strut‑to‑platform mount may permit rapid and reversible transition between dynamic hexapod operation and rigid reduction operation using a single joint assembly 36E, without requiring removal or replacement of the strut assembly 22.

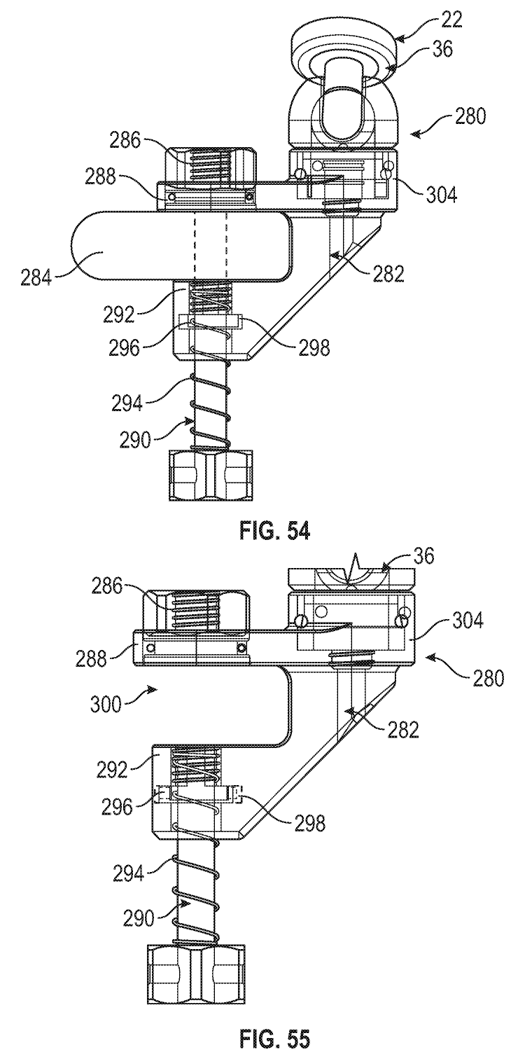

FIGS. 54–58 illustrate an exemplary quick‑connect mount 280 for mounting the strut assembly 22 to a platform 284 of the external fixation system 20. The quick‑connect mount 280 may enable rapid, tool‑free attachment and detachment of the strut assembly 22 relative to the external fixation system 20.

The quick‑connect mount 280 may include a mount body 282 configured to releasably engage the platform 284, a lock nut 286 rotationally retained within an opening formed in an upper platform 288 of the mount body 282, and a lock screw 290 operably coupled to a lower platform 292 of the mount body 282. The lock screw 290 may be biased toward an extended position relative to the mount body 282 by a biasing member 294, such as a spring, such that the lock screw 290 and the lock nut 286 remain operatively engaged during handling and assembly.

The upper platform 288 of the mount body 282 may further include a mount base 304 configured to facilitate attachment of a joint assembly 36 of the strut assembly 22 to the quick‑connect mount 280. Any of the joint assembly configurations described herein may be mounted to the mount base 304, thereby allowing the quick‑connect mount 280 to be used with a variety of strut‑to‑platform articulation mechanisms.

A cross pin 296 may be secured to the lock screw 290 and is movably received within a groove 298 formed inside the lower platform 292 of the mount body 282. In a first position, the cross pin 296 may be aligned within the groove 298 to permit lateral insertion of the platform 284 into a slot 300 formed in the mount body 282. Following insertion of the platform 284, rotation of the lock screw 290 may reposition the cross pin 296 out of alignment with the groove 298 and into alignment with an exit slot 302, thereby enabling axial movement of the lock screw 290 toward the lock nut 286 and capturing the platform 284 within the slot 300.

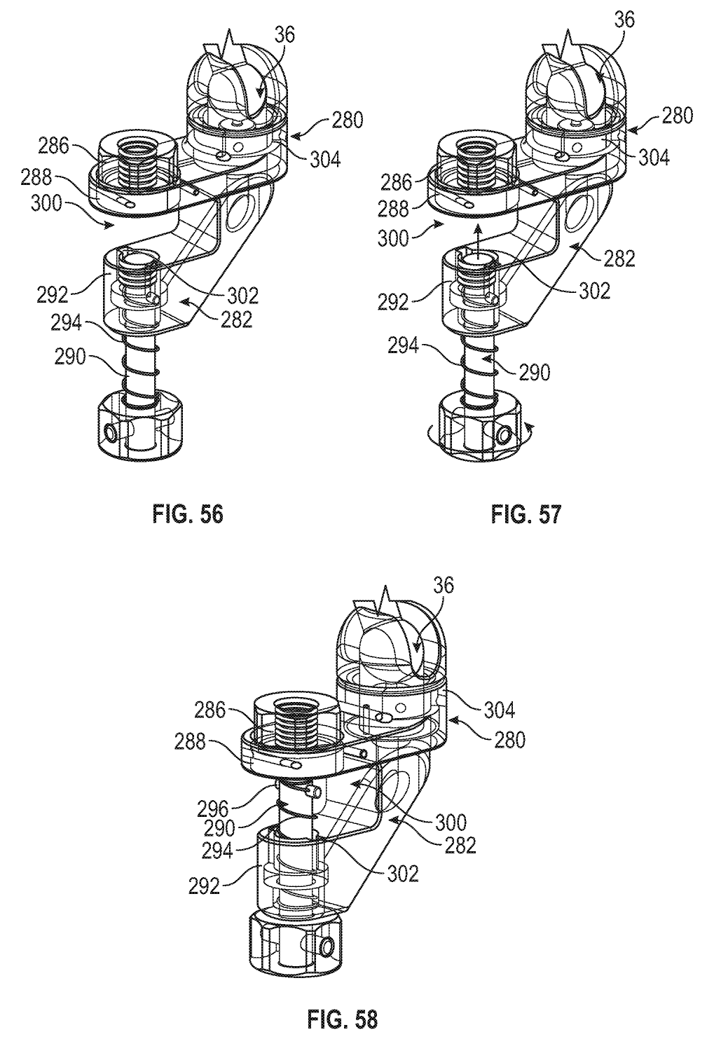

Upon release of the lock screw 290, the biasing member 294 may urge the lock screw 290 toward the lock nut 286. Subsequent rotation of the lock screw 290 may draw the mount body 282 into clamping engagement with the platform 284. Continued rotation may fully secure the strut assembly 22 relative to the platform 284. To detach the strut assembly 22, the lock screw 290 may be pulled back against the biasing force of the biasing member 294 and rotated to realign the cross pin 296 with the exit slot 302, thereby allowing removal of the platform 284 from the mount body 282. An exemplary connection sequence is schematically illustrated in FIGS. 56–58.

In this manner, the quick‑connect mount 280 may provide a compact and intuitive attachment mechanism that reduces assembly time and facilitates rapid frame construction or adjustment while maintaining reliable load transfer and secure fixation of the strut assembly 22 during treatment.

It should be understood that the various features, structures, and mechanisms disclosed herein may be implemented independently of one another or in any combination, unless expressly stated otherwise. By way of example, and without limitation, the strut assembly 22 may include any one or more of an incremental tactile feedback mechanism, a macro quick‑adjust mechanism, one or more modular or cartridge‑style extension components, one or more joint assemblies including articulating or lockable joint configurations, a dynamization mechanism providing controlled axial compliance, and one or more ring‑mount or quick‑connect mounting interfaces. Any such feature may be provided alone or combined with any other disclosed feature or features in a single strut assembly or fixation construct. The disclosure therefore contemplates all individual features as well as all permutations and combinations thereof, whether or not explicitly described, and no feature is intended to be required in any embodiment absent express recitation.

The exemplary universal strut assemblies of this disclosure provide a versatile, modular, and user-friendly solution for external fixation and deformity correction. The disclosed designs enable precise incremental adjustment and rapid macro length changes using tactile feedback mechanisms that improve repeatability and reduce user error. Modular threaded rod architectures and selectively attachable add-on components allow a wide range of strut lengths to be accommodated with reduced inventory complexity. Multiple strut-to-ring mount configurations, including swivel, quick-connect, lockable, dynamically adjustable, and convertible hexapod/reduction mounts, offer flexibility in surgical setup, postoperative adjustment, and frame rigidity, while enabling tool-free or minimally tooled operation. Integrated locking, detent, and dynamization features permit controlled angular positioning, selective rigid fixation, and adjustable frame compliance to support bone healing protocols. Collectively, these features enhance surgical efficiency, postoperative usability, patient comfort, and treatment precision while maintaining robust load-bearing performance across a variety of clinical configurations.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A strut assembly for an external fixation system, comprising:
a strut body extending along a strut axis;
a joint assembly connected to the strut body; and
a dynamization mechanism operably arranged between the strut body and the joint assembly and configured to selectively control axial compliance of the strut assembly along the strut axis,
wherein the dynamization mechanism is configurable between (i) a locked mode in which the strut assembly resists an axial length change along the strut axis under load, and (ii) an unlocked mode in which the strut assembly permits a controlled axial displacement along the strut axis under load while biasing the strut assembly toward a predefined length upon a reduction of the load.

2. The strut assembly as recited in claim 1, wherein the dynamization mechanism includes a dynamization shaft and a dynamization adjustment control that is operably connected to the dynamization shaft.

3. The strut assembly as recited in claim 2, wherein a first end of the dynamization shaft is secured to the joint assembly, and a second end of the dynamization shaft is secured to a coupler that is secured to the strut body.

4. The strut assembly as recited in claim 3, wherein the first end of the dynamization shaft is secured to a swivel housing of the joint assembly.

5. The strut assembly as recited in claim 2, wherein the dynamization adjustment control includes a user-actuatable adjustment knob that is rotatable to transition the dynamization mechanism between the locked mode and the unlocked mode.

6. The strut assembly as recited in claim 5, wherein the user-actuatable adjustment knob threadably engages the dynamization shaft.

7. The strut assembly as recited in claim 5, wherein rotation of the user-actuatable adjustment knob about the strut axis is configured to vary the controlled axial displacement in the unlocked mode.

8. The strut assembly as recited in claim 1, wherein the dynamization mechanism includes a biasing member arranged to urge the strut assembly toward the predefined length upon the reduction of the load when the dynamization mechanism is in the unlocked mode.

9. The strut assembly as recited in claim 8, wherein the biasing member is arranged between a dynamization adjustment control and a coupler of the dynamization mechanism.

10. The strut assembly as recited in claim 1, wherein the dynamization mechanism includes a retention pin movable within a retention slot extending parallel to the strut axis.

11. The strut assembly as recited in claim 10, wherein the retention pin secures a coupler of the dynamization mechanism to the strut body, and the retention slot is formed in the strut body.

12. The strut assembly as recited in claim 10, wherein the retention slot is sized to delimit a maximum permitted axial displacement of the strut assembly in the unlocked mode.

13. The strut assembly as recited in claim 12, wherein the retention pin is configured to engage an end of the retention slot to limit axial displacement when the maximum permitted axial displacement is reached.

14. The strut assembly as recited in claim 1, wherein the controlled axial displacement is enabled in the unlocked mode without removal of the strut assembly from the external fixation system.

15. The strut assembly as recited in claim 1, wherein the locked mode is configured for use during an initial post‑operative fixation phase and the unlocked mode is configured for use during a subsequent bone‑healing phase.

16. A strut assembly for an external fixation system, comprising:
a strut body;
a threaded rod assembly translatably received within an internal cavity of the strut body to adjust an axial length of the strut assembly; and
a joint assembly connected to the threaded rod assembly or the strut body, the joint assembly including:
a ball joint configured to permit angular articulation; and
a locking mechanism operably associated with the ball joint and configured to selectively restrict relative angular movement of the ball joint.

17. The strut assembly as recited in claim 16, wherein the locking mechanism is configured to permit partial locking of the ball joint to allow limited angular movement under load.

18. The strut assembly as recited in claim 17, wherein the limited angular movement provides controlled dynamization of the strut assembly.

19. The strut assembly as recited in claim 16, wherein the locking mechanism is adjustable between an unlocked state permitting angular articulation of the ball joint and a locked state restricting the angular articulation of the ball joint.

20. The strut assembly as recited in claim 16, wherein the ball joint includes a swivel lock cap and a ball portion movably received by the swivel lock cap.
